(19) United States
(12) Patent Application Publication
(10) Pub. No.: US 2026/0203626 A1
(43) Pub. Date: Jul. 16, 2026

(54) APPARATUS FOR GENERATING CONTROL SIGNALS FOR MEASURING STATES OF QUANTUM ELEMENTS OF A QUANTUM COMPUTER

(71) Applicants: BASF SE, Ludwigshafen am Rhein (DE); Robert Bosch GmbH, Stuttgart (DE)

(72) Inventors: Michael KUEHN, Ludwigshafen am Rhein (DE); Peter PINSKI, Karlsruhe (DE); Sebastian ZANKER, Karlsruhe (DE); Jan-Michael REINER, Karlsruhe (DE); Nicolas VOGT, Karlsruhe (DE); Thomas ECKL, Renningen (DE)

(21) Appl. No.: 19/132,318

(22) PCT Filed: Nov. 17, 2023
(86) PCT No.: PCT/EP2023/082160

§ 371 Date: May 22, 2025

(30) Foreign Application Priority Data
Nov. 22, 2022 (EP) 22208949.2

(51) Int. Cl. G06N 10/40; G06N 10/20; G06N 10/60

(52) U.S. Cl. CPC G06N 10/40; G06N 10/20; G06N 10/60

(57) ABSTRACT

The invention refers to an apparatus 810 for generating control signals for measuring states of quantum elements of a quantum computer 830. A providing unit 811 provides a problem description. A transformation unit 812 transforms the problem description into a quantum mechanical representation The transformation comprises determining unitary transformations that rotate operators representing observables to be measured into bases that lead to a pure occupation number representation. A translation unit 813 translates the quantum mechanical representation into a sequence of quantum operations comprising unitary transformation operations to be applied to quantum elements. The translating comprises determining the unitary transformation operations based on the determined unitary transformations. A generation unit 814 generates signals for controlling the application of the determined sequence of quantum operations to the quantum computer such that the quantum mechanical representation of the problem is prepared and the observables indicative of the solution of the problem are measured.

### FIELD OF THE INVENTION

The invention relates to an apparatus, a method and a computer program product for generating control signals for measuring states of quantum elements of a quantum computer. Moreover, the invention refers to a system for performing a quantum mechanical calculation on a quantum computer using the control signals. Further, the invention refers to an apparatus, a method and a computer program product for determining a solution of a problem utilizing the above apparatus, method and/or computer program product for generating control signals for measuring states of quantum elements of a quantum computer. Moreover, the invention relates to an apparatus, method and computer program product for determining target technical application properties of a chemical product utilizing the above method, apparatus and/or computer program product.

### BACKGROUND OF THE INVENTION

Quantum computers generally are a completely new kind of computing system that allows utilizing the special behaviour of quantum mechanical systems for performing problem calculations that, under the right circumstances, are not performable by ordinary computers in any reasonable time, or with reasonable resources. Moreover, it has already been shown that quantum computers are especially suitable for solving problems that can be related to the quantum mechanical world, i.e., problems that can be translated into a quantum mechanical description. Such problems relate, for instance, to electronic structure problems, molecular problems, condensed matter problems, etc. Solutions of such problems are in particular helpful in the context of chemical product optimization and designing. However, also problems outside of the description of physical quantum mechanical systems can be translated into a quantum mechanical description, for example, coding and decoding problems, complex analysis problems, optimization problems, etc. Examples for the translation of such problems can be generally found, for example, in the article “Quantum algorithms: an overview” by A. Montanaro, A., npj Quantum Information, volume 2, pages 1 to 8 (2016).

However, a drawback of quantum computers is that for determining a solution of a problem not only the state of each quantum element has to be measured by applying respective control pulses followed by a respective hardware-specific readout protocol but further this quantum mechanical measurement is inherently a statistical process such that for obtaining a meaningful result of a quantum computer calculation the measurements for each quantum element have to be averaged over a plurality of repeated state preparations. The number of required measurements typically increases strongly with the size of the problem and the required accuracy of the result, such that the effort of performing these measurements can strongly limit the applicability of a quantum computer calculation to problems with larger problem sizes. Thus, it would be advantageous if a possibility could be provided that decreases the efforts, i.e. the amount of measurements, necessary for measuring a result of a quantum computer calculation. Such a possibility would allow to apply quantum computer calculations to problems important for chemical applications and allow to solve problems in this context not only faster and with less computational resources but also more accurately.

### SUMMARY OF THE INVENTION

It is an object of the present invention to provide apparatuses, methods, systems and computer program products that allow to decrease the effort, in particular, the number of state preparations, necessary for providing a solution of a quantum computer calculation, in particular, a solution of a problem being translatable into a quantum mechanical description utilizing a quantum computer. Moreover, the apparatuses, systems, methods and computer program products allow for the calculation of problems related to chemical products or solid product comprising an increased complexity and/or problem size allowing for a wider application of quantum computers in this technical field that can lead to providing solutions for the chemical industries that are more accurate, provided faster and with less computational resources.

In a first aspect of the present invention, an apparatus for generating control signals for measuring states of quantum elements of a quantum computer is presented, wherein the measured states are indicative of observables indicative of a solution of a problem being translatable into a quantum mechanical description, wherein the apparatus comprises i) a problem providing unit for providing a problem description indicative of the problem to be solved, wherein the problem description is translatable into a quantum mechanical description, ii) a transformation unit for transforming the problem description into a quantum mechanical representation comprising one or more operators representing one or more observables to be measured indicative of the solution of the problem, wherein the transformation further comprises determining unitary transformations that rotate the one or more operators representing one or more observables to be measured into one or more bases that lead to a pure occupation number representation of the one or more operators after application of the unitary transformations, iii) a translation unit for translating the quantum mechanical representation into a quantum algorithm description comprising a sequence of quantum operations to be applied to quantum elements of the quantum computer, wherein the sequence of quantum operations comprises a) a preparation part comprising quantum operations for preparing the quantum mechanical representation on the quantum computer such that the observables being indicative of the solution of the problem are measurable, and b) a measuring part comprising quantum operations for measuring the observables by measuring the states of the quantum elements after the preparation of the quantum mechanical representation, wherein the measurement operations comprise unitary transformation operations to be applied to quantum elements, wherein the translating comprises determining the unitary transformation operations based on the determined unitary transformations such that the unitary transformation operations initiate a rotation of the states of the quantum elements into respective basis states corresponding to the one or more bases leading to the pure occupation number representation of the one or more operators representing the one or more observables to be measured, and iv) a control signal generation unit for generating control signals for controlling the application of the determined sequence of quantum operations to the quantum computer such that the quantum mechanical representation of the problem is prepared and the observables indicative of the solution of the problem are measured according to the quantum algorithm description.

Since the representation of the problem description is transformed by the measurement operations comprising unitary transformation operations initiating a rotation of the states of the quantum elements into basis states corresponding to the one or more bases leading to a pure occupation number representation of operators representing the observables to be measured, the states of the quantum elements representing the observables can be measured concurrently. This strongly reduces the number of necessary state preparations and measurements, in particular non-concurrent measurements, for determining a result of a quantum computer calculation. Thus, the calculation of larger problems, for example, larger molecules, more accurate calculations, for example, taking into account more variables, or a faster and less resource-intensive calculation of smaller problems becomes possible.

Generally, the apparatus can be realized in form of software or hardware or a combination thereof, wherein the hardware can refer to any known dedicated or general classical computer hardware. For example, the apparatus can be realized as any known computational device, like a PC. However, the apparatus can also be realized as a cloud environment, computational network, etc., such that at least parts of the apparatus can also be realized as a network solution and thus can be spread over a plurality of computational devices. The apparatus is adapted to provide control signals that can be provided to, i.e. are interpretable by, any known quantum computer hardware architecture.

The problem can generally refer to any problem that is translatable into a quantum mechanical description and thus can be calculated on a quantum computer. However, it is preferred that the problem is related to a chemical product or solid product, for example, a solution of the problem can be utilized for determining a technical application property of a chemical product or the solution of the problem can be utilized for optimizing a chemical reaction for producing a chemical product. Examples for such problems that can advantageously be solved in this invention can refer to calculating a ground state energy of a molecule or general electronic system. This allows, in particular, in the context of determining the ground state energies of all molecular species occurring in a chemical reaction to predict the reaction end products, the thermodynamic properties of the reaction, and the kinetic properties of the reaction. This understanding and the properties of the reaction can then again be utilized to optimize chemical production processes, for prediction of a microstructure of polymers, for optimizing material properties, etc. Further, an important problem refers to determining a ground state of a solid. This allows, for instance, to predict magneto-crystalline anisotropies of magnetic materials which are important for magnetic materials, for example, in an electric motor. Moreover, the problem can also refer to calculating multipole moments of a chemical product. Such a calculation can be relevant for determining properties of the chemical product related to electrical properties, e.g. the behavior of the chemical product in an electrical field. Moreover, it is preferred that the problem is translatable into a quantum mechanical description referring to a fermion system, in particular, an electronic structure problem, since for such problems the method performed by the apparatus is in particular suitable and allows for a particular decrease of the number of necessary state preparations and non-concurrent measurements.

The problem providing unit is adapted to provide a problem description indicative of the problem being translatable into a quantum mechanical description. In particular, the problem providing unit can refer to a storage unit on which the problem description is already stored. However, the problem providing unit can also comprise an input unit with which, for instance, a user can indicate a problem description of the problem to the problem providing unit. The problem description can refer to any form of description of the problem that allows to determine the quantities describing the problem and the form of interaction between these quantities. Preferably, the problem description refers to a mathematical description of the problem. However, the problem description can also refer to any other unambiguous form of notation of the problem. In a preferred embodiment, the problem description is already provided in form of a quantum mechanical description, wherein a quantum mechanical description represents the problem in terms of quantities following the quantum mechanical rules, i.e. refers to a representation of the problem in the quantum mechanical world. However, the problem description can also be provided in any other form, wherein in this case it is preferred that the providing unit is adapted to translate the provided problem description into a quantum mechanical problem description before providing the problem description to the transformation unit. However, this translation can also be omitted, wherein in this case the transformation unit and the translation unit are preferably adapted to process the respective form of the problem description accordingly, for instance, by utilizing principles derived from the processing of the problem description in the quantum mechanical description or to translate the problem description accordingly.

The transformation unit is configured to transform the problem description into a quantum mechanical representation comprising one or more operators representing one or more observables to be measured. In particular, the quantum mechanical representation refers to the representation of at least a part of the quantum mechanical description of the problem description that can be prepared on a quantum computer and allows to determine a solution of the problem based on the measured states of the quantum elements of the quantum computer. Generally, depending on the problems, methods for transforming a respective problem into a quantum mechanical representation being prepared on a quantum computer for determining a solution of the problem are well-known. Examples of these known methods for some general and specific problems can be found in the article “Quantum Computational Chemistry”, S. McArdle et. al., Rev. Mod. Phys. 92, (2020), “Quantum Algorithms for Quantum Chemistry and Quantum Materials Science”, B. Bauer et. al., Chem. Rev. (2020), and “Quantum Chemistry in the Age of Quantum Computing”, Y. Cao et al., Chem. Rev. (2019). In particular, the quantum mechanical representation comprises one or more operators, i.e. quantum mechanical operators, representing the one or more observables to be measured. Generally, an observable refers to a measurable quantity, for instance, a position, momentum, energy, magnetic moment, etc. and is associated in quantum mechanics with an operator, for example, the observable energy is associated with a Hamiltonian operator, that represents the observable in a Hilbert space in which a quantum mechanical representation is formulated. The transformation comprises not only determining a general quantum mechanical representation that can be prepared on the quantum computer, but further determining unitary transformations that rotate the one or more operators representing the one or more observables to be measured into one or more bases that lead to a pure occupation number representation of the one or more operators after application of the unitary transformations. Generally, unitary transformations refer to transformations that preserve an inner product of two quantities. In particular, the unitary transformations change the basis of the one or more operators, but do not change the observable represented by the operators. Accordingly, after the application of the unitary transformations still the same value for the observable will be measured. Providing a transformation of the problem description that results in a quantum mechanical representation comprising operators in a pure occupation number representation has the advantage that the measurements of the observables represented by these operators on the quantum computer can be performed concurrently. Generally, a pure occupation number representation refers to a problem description comprising observables that can be measured concurrently on a quantum computer. To be measurable concurrently observables have a quantum mechanical representation where i) the observables are compatible, i.e., they have a common basis in the Hilbert space which comprises the quantum mechanical description, i.e., the operators representing the observables commute, and ii) the Pauli operators representing the observables have a common measurement basis. An example are number operators of electrons that can be represented by Pauli-Z operators using a Jordan-Wigner transform and which then fulfill both requirements.

Generally, in case the problem description is not provided in form of a quantum mechanical description, the transformation unit is preferably further adapted to translate the problem description accordingly before determining the quantum mechanical representation, e.g. to map the problem description to a description of a quantum mechanical system that can generally be simulated by the respective chosen quantum computer, for example, by mapping the problem description to a Hamiltonian of a quantum mechanical system that defines similar relations between and influences on quantities as the problem description. Thus, for instance, an optimization problem referring to the field of optimizing production parameters for producing a product, like temperature, pressure, flow velocity, etc., can be translated into a quantum mechanical description representing the problem in the quantum mechanical world of the quantum computer. In such a case, for instance, Ising models can be utilized for the translation. However, if the problem already refers to a quantum mechanical problem, for instance, an electronic structure problem, this particular step of translating the problem into a quantum mechanical description can be omitted. Generally, the transformation unit can be adapted to translate the problem description based on predetermined rules or a predetermined model for specific problem categories or can be adapted to translate the problem description in an interactive process based on user input. In the case of an interactive process the user can, for instance, be provided with a user interface that allows the user to select different problem categories, like, optimization problem, electronic structure problem, etc. to determine the category of the provided problem and can further select a respective set of rules or a model that shall be applied for translating the provided problem. However, also other interaction can be facilitated by a user interface for translating the problem. Moreover, for translating the provided problem, the translation unit can also be adapted to access a storage on which translations for specific problems are already stored, for instance, for problems that have already been solved before, e.g. for different parameters.

The translation unit is then configured to translate the quantum mechanical representation of the problem into a quantum algorithm description comprising a sequence of quantum operations to be applied to quantum elements of the quantum computer. The term “quantum element” may refer to the fundamental building block of a quantum computer. In the context of quantum computing, these elements may be commonly known as qubits. Qubits may be the quantum analog of classical bits. While classical bits can represent either a 0 or a 1, qubits may exist in a superposition of both states simultaneously, thanks to the principles of quantum mechanics. Qubits may be realized using various physical systems, such as atoms, ions, superconducting circuits, or photons. These physical systems may provide a way to manipulate and measure the quantum state of a qubit.

Generally, operations are performed by a quantum computer by manipulating the states of quantum elements of the quantum computer, wherein the quantum elements can refer to all elements of the quantum computer that are utilized for simulating the problem, for instance, to quantum elements forming the qubits of a quantum computer. The sequence of quantum operations determined by the translation unit comprises a preparation part and a measuring part. Generally, the preparation part comprises quantum operations for preparing the quantum mechanical representation of the problem on the quantum computer such that the observables being indicative of the solution of the problem are measurable. General methods for determining such operations for problems translatable into quantum mechanical representations are generally known and can be utilized by the translation unit for determining the preparation part of the quantum operations. Examples of these known methods for some general and specific problems can be found in the article “Quantum Computational Chemistry”, S. McArdle et. al., Rev. Mod. Phys. 92, (2020), “Quantum Algorithms for Quantum Chemistry and Quantum Materials Science”, B. Bauer et. al., Chem. Rev. (2020), and “Quantum Chemistry in the Age of Quantum Computing”, Y. Cao, et al., Chem. Rev. (2019).

The measuring part of the quantum operations comprises quantum operations for measuring the observables of the quantum mechanical representation by measuring the states of the quantum elements after the preparation of the quantum mechanical representation. These measurement operations comprise unitary transformation operations to be applied to the quantum elements. The unitary transformation operations are determined based on the unitary transformations determined by the transformation unit such that the unitary transformation operations initiate a rotation of the states of the quantum elements into respective basis states corresponding to the one or more bases that lead to the pure occupation number representation of the one or more operators representing the one or more observables to be measured as determined by the transformation unit. Accordingly, the unitary transformations determined by the transformation unit are utilized by the translation unit for determining the unitary transformation operations and for bringing the quantum elements that are in the states indicated by the quantum mechanical representation, in particular, in the states after the application of the preparation part of the sequence of operations, into a state that represents the pure occupation number representation of the one or more operators representing the one or more observables to be measured. Generally, the application of the unitary transformation operations changes the states of the quantum elements such that the states of the quantum elements represent the observables in the bases leading to the pure occupation number representation. However, the measurement of the states of the quantum elements provides the same results with respect to the solution of the problem, i.e. does not change the to be determined value of the observables. However, changing the basis allows for reading out, i.e. measuring, the states of the quantum elements concurrently based on the same preparation of the quantum mechanical representation on the quantum computer instead of requiring several preparations of the quantum computer for a measurement of the observables represented by the states of the quantum elements for each observable again. This allows to strongly decrease the number of necessary preparations for measurements during a quantum computer calculation.

The control signal generation unit is configured to generate control signals for controlling the application of the determined sequence of quantum operations to the quantum computer. In particular, the control signals are generated such that the quantum mechanical representation of the problem is prepared on the quantum computer and the observables indicative of the solution of the problem are measured according to the quantum algorithm description. In particular, the control signals can refer to signals that directly control a quantum computer, for instance, a manipulation unit of a quantum computer for manipulating the states of the quantum elements, but can also refer to an indirect control of the quantum computer, for example, by providing the control signals to a quantum computer controller that then translates the control signals into respective control actions of the manipulation parts of the quantum computer. Thus, the control signals can be provided in any format that is directly utilizable for controlling a quantum computer or that is readable by a quantum computer controller and can then be translated into respective control actions.

In an embodiment, the transformation unit is adapted to determine the unitary transformations by applying i) a factorization to a tensor representation of one or more operators of the one or more observables to be measured and ii) a diagonalization to the resulting matrix terms of the factorized tensor representation of the one or more operators. In most application cases, at least some of the operators representing the observables can be represented as tensors. A respective matrix factorization can then be utilized to decompose the tensor into sum over products of matrices. Thus, through the factorization a tensor representing one or more operators is decomposed into a product of matrix terms. These matrix terms of the factorized tensor representation can then be diagonalized for determining the unitary transformations based on the diagonalized matrix terms. Generally, known algorithms can be utilized by the transformation unit for performing the factorization and the diagonalization. Preferably, the transformation unit is adapted, for the diagonalization of the matrix terms in the factorized tensor representation of the one or more operators, to determine, for each matrix term of the factorized tensor representation, eigenvalues and corresponding eigenvectors and to determine the to be measured observables as an expression in terms of corresponding eigenvectors and eigenvalues of the factorized tensor representations of the operators. Thus, the factorization and diagonalization allow to represent the operators representing the observables in terms of eigenvalues and corresponding eigenvectors. In particular, the bases to which the operators are rotated by the unitary transformations refer to the eigenvector states of the factorized tensor representations of the operators. Accordingly, the unitary transformation operations can be determined by determining the operations that allow to rotate the quantum elements to states that refer to the respectively determined eigenvector states of the factorized tensor representations.

In a preferred embodiment, the problem description is translatable into a relativistic Hamiltonian description of the problem. In this case, it is preferred that the transformation unit is adapted to apply a Takagi factorization for determining the unitary transformations. Alternatively, it is preferred that the transformation unit is adapted to apply a pivoted Cholesky factorization for determining the unitary transformations. In particular, both factorization methods can be applied by the transformation unit for factorizing the tensor representation of the one or more operators, for example, as described above. Moreover, it is preferred that these two factorization methods are in particular applied to operators referring to two-fermion operators, in particular, two-electron operators, of a relativistic Hamiltonian.

In an embodiment, the transformation unit is adapted to separate operators of the quantum mechanical representation representing observables to be measured into a first part comprising first operators and a second part comprising second operators, wherein the transformation unit is adapted to transform the second operators into a quantum mechanical representation comprising only a pure occupation number representation of the operators representing the one or more observables to be measured. By separating the operators representing the one or more observables to be measured into a first part and a second part and transforming only the second operators into a pure occupation number representation, for example, utilizing any of the methods described above, the transformation is only performed, for instance, on terms, i.e. operators, of the quantum mechanical representation for which the transformation is most advantageous. In particular, the first part and the second part are determined such that transforming the second part allows to decrease the number of necessary preparations for measurements, whereas, for example, the transformation of the first part would not provide such further benefits, i.e. would not result in a further reduction of the number of the necessary preparations for measurements. Thus, by determining the first part and the second part accordingly, less computational resources are necessary for the transformation and determination of the unitary transformations. Preferably, the first operators of the first part are translatable into a quantum mechanical representation that refers to a pure occupation number representation of the operators representing the observables to be measured. Thus, if possible, it is preferred that the first part refers to a part of the operators that is already translatable into a pure occupation number representation without applying a respective transformation as described above. Thus, for this part it is not necessary to apply unitary transformations for allowing for an improved measurement of the states of the quantum elements.

In a preferred embodiment the quantum mechanical representation comprises two-electron operators, wherein the transformation unit is adapted to transform the two-electron operators into terms of a density-density interaction. Generally, a representation transformed into terms of a density-density interaction is an example for an occupation number representation, such that for transforming the two-electron operators into terms of a density-density interaction also the methods, in particular, the factorization and diagonalization leading to a determination of the eigenvalues and eigenvectors, described above can also be utilized. However, also other methods can be utilized that allow to transform two-electron operators into terms of a density-density interaction. Preferably, the transformation unit is adapted to transform the two-electron operators into terms of a density-density interaction by utilizing a resolution of the identity (RI) approximation. This method allows for a particular efficient measurement of two-electron operators on a quantum computer.

In an embodiment, the quantum mechanical representation comprises operators referring to an arbitrary-order reduced density matrix, in particular, operators with an expectation value referring to an arbitrary-order reduced density matrix, and an arbitrary-order tensor, wherein the transformation unit is adapted to symmetrize and/or hermitize the arbitrary-order tensor before determining the unitary transformations. In particular, many problems relevant for chemical applications comprise such operators as defined above. Symmetrizing and/or hermitizing this tensor then allows to utilize, for example, the above described methods for determining the unitary transformations. Accordingly, the symmetrizing and/or hermitizing of the respective tensor allows to increase the range of problems for which a respective improved measurement can be performed. Preferably, the transformation unit is adapted to further apply a low-rank decomposition to the symmetrized and/or hermitized arbitrary-order tensor for determining the unitary transformations. The low-rank decomposition can, for instance, comprise the steps of factorization and diagonalization and determining of the eigenvectors and eigenvalues as already described above.

In a further aspect of the invention, a system for performing a quantum mechanical calculation on a quantum computer is presented, wherein the system comprises i) a quantum computer adapted to perform quantum mechanical calculations based on provided control signals, and ii) an apparatus according to any of the preceding claims adapted to provide control signals to the quantum computer for controlling the performance of a quantum mechanical calculation.

In a further aspect of the invention, a computer implemented method for generating control signals for measuring states of quantum elements of a quantum computer is presented, wherein the measured states are indicative of observables indicative of a solution of a problem being translatable into a quantum mechanical description, wherein the method comprises i) providing a problem description indicative of the problem to be solved, wherein the problem description is translatable into a quantum mechanical description, ii) transforming the problem description into a quantum mechanical representation comprising one or more operators representing one or more observables to be measured indicative of the solution of the problem, wherein the transformation further comprises determining unitary transformations that rotate the one or more operators representing one or more observables to be measured into one or more bases that lead to a pure occupation number representation of the one or more operators after application of the unitary transformations, iii) translating the quantum mechanical description into a quantum algorithm description comprising a sequence of quantum operations to be applied to quantum elements of the quantum computer, wherein the sequence of quantum operations comprises a) a preparation part comprising quantum operations for preparing the quantum mechanical representation on the quantum computer such that the observables being indicative of the solution of the problem are measurable, and b) a measuring part comprising quantum operations for measuring the observables by measuring the states of the quantum elements after the preparation of the quantum mechanical representation, wherein the measurement operations comprise unitary transformation operations to be applied to quantum elements, wherein the translating comprises determining the unitary transformation operations based on the determined unitary transformations such that the unitary transformation operations initiate a rotation of the states of the quantum elements into respective basis states corresponding to the one or more bases leading to the pure occupation number representation of the one or more operators representing the one or more observables to be measured, and iv) providing control signals for controlling the application of the determined sequence of quantum operations to the quantum computer such that the quantum mechanical representation of the problem is prepared and the observables indicative of the solution of the problem are measured according to the quantum algorithm description.

In a further aspect of the invention, a computer program product for generating control signals for measuring a state of quantum elements of a quantum computer is presented, wherein the computer program product comprises program code means for causing the apparatus or system as described above, respectively, to execute the method as described above.

In a further aspect of the invention, a solution apparatus for determining a solution of a problem being translatable into a quantum mechanical description is presented, wherein the apparatus comprises i) an apparatus as described above for generating control signals for controlling a quantum computer, ii) a quantum computer interface unit for interfacing with a quantum computer for providing the control signals to the quantum computer and receiving a result of the measured observables, and iii) a determination unit configured for determining a solution of the problem based on the received measurement results.

Generally, the solution of the problem based on the received measurement results can be determined in any known way, in particular, depending on the specific problem and, in particular, can be performed on a classical computing system. Moreover, for determining the solution of the problem, also a hybrid computing approach can be utilized in which, based on the result of the measurement of the observables on a quantum computing system, computations on classical computing systems are performed and the result of these classical computations is again utilized as input into a quantum computer calculation, for example, by amending one or more variables in the problem solved on the quantum computer, and again results of the measurements of the observables are utilized for further calculations on a classical computing system. Such an iteration can be utilized to increase the accuracy of a solution of a problem while at the same time optimizing the required computational resources for the calculation of the problem.

In a further aspect of the invention, a computer implemented solution method for determining a solution of a problem being translatable into a quantum mechanical description is presented, wherein the method comprises i) using any one of the apparatuses as described above, the method as described above and the computer program product as described above for generating control signals for controlling a quantum computer, ii) interfacing with a quantum computer for providing the control signals to the quantum computer and receiving a result of the measured observables, and iii) determining a solution of the problem based on the received measurement results.

In a further aspect of the invention, a property determination apparatus for determining a technical application property of a chemical product or solid product based on a solution of a problem related to the chemical product or solid product is presented, wherein the problem is translatable into a quantum mechanical description, wherein the apparatus comprises i) an apparatus as described above for generating control signals for controlling a quantum computer, ii) a quantum computer interface unit for interfacing with a quantum computer for providing the control signals to the quantum computer and receiving a result of the measured observables, and iii) one or more processors configured for determining the technical application property of the chemical product or solid product based on the received measurement results. Generally, the chemical product can refer to any chemical product, for example, can refer to a substance or a mixture of substances, a molecule, a protein, a polymer, etc. In particular, the chemical product can be any organic or anorganic chemical product, chemical molecule and/or biological. The solid product can be any solid product, for example, a metal compound, a crystal, etc. In particular the solid product may be solid with a periodic lattice structure. Preferably the solid product is a semiconductor or superconductor.

A technical application property can generally refer to any property of a chemical product or solid product that allows to assess a technical applicability of the respective chemical product or solid product as provided after its production. Preferably, the technical application property comprises at least one of mechanical properties, spectroscopic properties, physicochemical properties, chemical properties and biological properties. Generally, mechanical properties can refer to any of adhesion, tensile strength, stiffness, hardness, shrinkage, elongation, split tear, tear-strength, rebound, compressibility, abrasion, spillage, morphology, haptic properties, stress at break, elongation at break, granulometry and a degree of filling. A spectroscopic property can generally comprise any of coloration, turbidity, opaqueness, lucidity, reflection, appearance, absorption, scattering, color strength, colour hue, colour saturation, colour intensity, cloud point, matting degree, optical density, spectra, refractive index, IR spectra, Raman spectra, NMR spectra, ESR spectra, and UV/Vis spectra. Moreover, a physicochemical property can refer to any of density, viscosity, K-value, molar weight, dispersity, molar mass distribution, particle size distribution, solubility, partition coefficients, interfacial properties, surface tension, dispersibility, storage stability, odor, segregation, coagulation, electric conductivity, electric capacity, surface area, flow time, vapor pressure, VOC, solid content, hygroscopicity, magnetism, miscibility, thixotropy, phase transition properties, glass transition temperature, corrosion inhibition, solvent separation, aggregation, self-heating ability, impact sensitivity, loss on drying, angle of response, electrostatic charge, minimum film-forming temperature, charge density, electrostatic multipole moments, and thermal conductivity. The chemical property can comprise any of reaction thermodynamics, reaction kinetics, chemical resistance, reaction timing, demolding time, growing, hard/soft segment content, crystallinity, reaction temperature, reaction pressure, decomposition, thermal decomposition, photodegradation, acidity, pKa, pH, moisture/water content, flammability, burning rate, self-ignition, flash point, formation of flammable gases, reaction to fire, deflagration rate, residual monomer count, side product formation, degree of polymerization, salt content, temperature tolerance, oxidizing properties, reduction properties, reactivity, ash content, nonvolatile matter content, stability, chelating ability, calorific value, saponification value. Further, the biological property can comprise any of biodegradability, biological resistance, in particular, resistance against a pathogenic virus, bacterium, fungus, plant or animal or developmental stage of said pathogen, tolerance against environmental parameters, e.g. drought tolerance, resistance against enzymatic degradation, e.g. protease resistance, lipase resistance, amylase resistance, hydrolase resistance, pesticide resistance, toxicity, biotransformation, ecotoxicology, sensitization, in particular, allergenicity, bacterial count, enzyme activity, substrate specificity, cofactor dependence, product specificity, substrate and/or product inhibition, dissociation constant, Michaelis-Menten-kinetics values, activity/stability at or in different: pH, temperature, pressure, organic solvent concentration, carrier formulations, encapsulation formulations; distribution in environment, compartimentalisation, bioaccumulation, biological exposure LD50, mutagenicity.

Generally, in this embodiment, the problem for which the apparatus as described above generates control signals for controlling a quantum computing system can refer to any problem for which a solution can lead to deriving the respective technical application property of a chemical product or solid product, for example, one of the technical application properties mentioned above. In particular, the problem can refer to an electronic structure problem, wherein based on the solution of the electronic structure problem a plurality of further characteristics of a respective chemical product or solid product can be derived. Examples for such problems that can advantageously be solved in this invention can refer to calculating a ground state energy of a molecule or general electronic system. This allows, in particular, in the context of determining the ground state energy of all molecular species occurring in a chemical reaction to predict the reaction end products, the thermodynamic properties of the reaction, and the kinetic properties of the reaction. This understanding and the properties of the reaction can then again be utilized to optimize chemical production processes, for prediction of a microstructure of polymers, for optimizing material properties, etc. Further, an important problem refers to determining a ground state of a solid. This allows, for instance, to predict magneto-crystalline anisotropies of magnetic materials which are important for magnetic materials, for example, in an electric motor. Moreover, the problem can also refer to calculating multipole moments of a chemical product. Such a calculation can be relevant for determining properties of the chemical product related to electrical properties, e.g. the behavior of the chemical product in an electrical field. Accordingly, the determining of the value of the technical application property based on the result of the measurements of the observables is based on the respective technical application property that should be determined and further based on the information that is provided by the solution of the problem indicated by the result of the measurements of the observables.

In a further aspect of the invention, a computer implemented property determination method for determining a technical application property of a chemical product or solid product based on a solution of a problem related to the chemical product or solid product is presented, wherein the problem is translatable into a quantum mechanical description, wherein the method comprises i) using any one of the apparatuses as described above, the method as described above and the computer program product as described above for generating control signals for controlling a quantum computer, ii) interfacing with a quantum computer for providing the control signals to the quantum computer and receiving a result of the measured observables, and iii) determining the technical application property of the chemical product or solid product based on the received measurement results.

In a further aspect of the invention, an apparatus for determining a target chemical product or solid product comprising a target technical application property is presented, wherein the apparatus comprises i) an input interface configured to provide a target technical application property and a potential chemical product or solid product, and ii) an apparatus as described above for generating control signals for controlling a quantum computer based on the potential chemical product or solid product and the target technical application property, iii) a quantum computer interface unit for interfacing with a quantum computer for providing the control signals to the quantum computer and receiving a result for the to be measured observables, and iii) one or more processors configured for a) determining the technical application property of the potential chemical product or solid product based on the received measurement results, and b) compare the determined technical application property of the potential chemical product or solid product with the target technical application property and, based on the comparison, either I) determining the potential chemical product or solid product as the target chemical product or solid product, or II) providing a new potential chemical product or solid product and repeating the determination of the technical application property utilizing the new potential chemical product or solid product, and iv) an output interface configured to provide control signals for producing the determined target chemical product or solid product.

In particular, in this context the providing performed by the input interface can refer to receiving the target application property from an input of a user applying, for instance, a respective input interface and providing the same to the quantum computer interface. Moreover, the providing can also refer to accessing a storage unit on which a target application property is already stored and providing the same. Further, the providing can also comprise receiving a target application property, for instance, via a network connection from other sources and providing the received target application property. Generally, the target application property can refer to one target value, for instance, a specific hardness of a chemical product or solid product, or can refer to a value range that should be met by the chemical product or solid product. Moreover, the target application property can also refer to any kind of target function, for instance, a timely sequence of a property under a changing environmental condition, like a hardness under changing temperature conditions. Such more complex target application properties can be advantageous in cases in which the application of the chemical product or solid product comprises different environmental conditions, for example, different temperatures. The target chemical product or solid product then refers to a chemical product or solid product that meets the respective target technical application property within predetermined limits when provided in a respective form, for example, as pure substance, or mixture. In particular, the target chemical product or solid product provides the respective target technical application property when produced according to a corresponding recipe.

A potential chemical product or solid product can be provided in any digital representable format such that the potential chemical product or solid product and/or characteristics of the potential chemical product or solid product can be processed by the apparatus. Moreover, the providing of the potential chemical product or solid product can also comprise providing a respective problem for determining the technical application property of the potential chemical product or solid product. However, the respective problem can also be selected automatically, for example, by the apparatus based on the potential chemical product or solid product and the provided target technical application property. However, also a user can, for example, select a respective problem preferably based on a selection of a plurality of possible problems presented to the user based on the potential chemical product or solid product and/or the target technical application property.

The comparison of the determined technical application property with the target technical application property allows to determine whether the determined technical application property fulfils a predetermined criterion, for example, that the determined technical application property meets the target technical application property within predetermined limits. If such a criterion is fulfilled, the potential target chemical product or solid product is determined as the target chemical product or solid product, respectively, and the method proceeds to the next step. However, if the comparison indicates that the determined technical application property does not meet the target technical application property within the predetermined limits, a next iteration step utilizing a new potential chemical product or solid product can be performed. In particular, for each iteration step of the iteration a new potential chemical product or solid product is determined, preferably based on the previous potential chemical product or solid product, for instance, by amending one or more features of the previous chemical product or solid product, e.g. one or more constituents or other characteristics. However, a new potential chemical product or solid product can also be generated by arbitrarily choosing a new potential chemical product or solid product, for example, from a large amount of previously generated potential chemical products or solid product. Moreover, also more sophisticated methods can be utilized for selecting a new potential chemical product or solid product from a plurality of previously already generated potential chemical products or solid product. Based on the new potential chemical product or solid product, in each iteration step again the quantum computer is utilized for determining the technical application property and the such determined technical application property is again compared with the target technical application property such that the comparison can again lead to a further iteration step or if the respective criterion is fulfilled the respective new potential chemical product or solid product can be selected as the target chemical product or solid product. Moreover, also an additional abortion criterion for the iteration can be selected, for instance, a number of iteration steps can be determined before the iteration is aborted with a notification to a user that no target chemical product or solid product could be found for the respective target technical application property. However, alternatively, after a predetermined amount of iteration steps the method can further comprise amending the target technical application property, for instance, by increasing the predetermined limits around the target technical application property and to repeat the iteration while utilizing the increased limits during the comparison. This can allow to find a target chemical product or solid product that meets the target technical application property as much as possible, even if a meeting of the original goal might not be possible. After the target chemical product or solid product has been determined as described above, the target chemical product or solid product can be provided, for instance, to a user via an output unit. Preferably, a recipe of the target chemical product or solid product is utilized to generate control data that can be utilized for controlling a production system for producing the target chemical product or solid product.

In a further aspect of the invention, a computer implemented method for determining a target chemical product or solid product comprising a target technical application property is presented, wherein the method comprises i) providing a target technical application property and a potential chemical product or solid product, and ii) using any one of the apparatuses as described above, the method as described above and the computer program product as described above for generating control signals for controlling a quantum computer based on the potential chemical product or solid product, iii) interfacing with a quantum computer for providing the control signals to the quantum computer and receiving a result of the measured observables, and iv) determining the technical application property of the potential chemical product or solid product based on the received measurement results, and v) comparing the determined technical application property of the potential chemical product or solid product with the target technical application property and, based on the comparison, either a) determining the potential chemical product or solid product as the target chemical product or solid product, or b) providing a new potential chemical product or solid product and repeating the determination of the technical application property utilizing the new potential chemical product or solid product, and vi) providing control signals for producing the determined target chemical product or solid product.

In a further aspect of the invention, a use of an apparatus as described above for solving problems referring to at least one of a chemical reactivity, spectra and spectroscopic properties, and molecular properties derivable from electronic structure problem calculations of a chemical product or solid product is presented.

In a further aspect of the invention, a use of an apparatus as described above is presented, for solving problems referring to at least one of metalorganic compounds containing transition metals including lanthanides and actinides, chelating agents interacting with metals, catalysts, biomolecules with active centers, macromolecular systems and transition metal compounds in solution or embedded in an environment.

In a further aspect of the invention, a use of an apparatus as described above is presented for determining activation and/or reaction energies for a predetermined chemical reaction. Generally, an activation energy refers to an energy difference between a transition state and reactants. A reaction energy refers to an energy difference between products and reactants. The chemical reaction can be part of a complex reactive network, e.g. of a catalytic cycle. Utilizing an apparatus as described above is in particular advantageous in cases where at least one species of this reactive system a) contains one or several transition metal atoms, lanthanide atoms and/or actinide atoms with unpaired electrons or b) exhibits an electronic structure with a small energetic gap between occupied and unoccupied electron orbitals, i.e., an energetic gap that is equal or smaller than the gap of at least one of the molecules ozone, pentacene or para-quinodimethane calculated with the same electronic structure approach, i.e. same basis set, same self-consistent field (SCF) method, e.g. Hartree-Fock, etc. or c) exhibits a multi-reference diagnostic that exceeds a predetermined limit, e.g. T₁(CCSD) greater than 0.02 and/or D₁(CCSD) greater than 0.05 and/or D₁(MP2) greater than 0.04 and/or D₂(MP2/CCSD) greater than 0.18 and/or Zₛ₍₁₎ greater than 0.1 and/or % TAE greater than 10, wherein the T₁ diagnostic is determined based on the Frobenius norm of the amplitudes for single excitations of a CCSD wavefunction based on a Hartree-Fock reference state scaled by the square root of the number of correlated electrons in the CCSD calculation, and wherein the D₁ diagnostic is determined based on the matrix 2-norm of the amplitudes for single excitations of a CCSD or MP2 wavefunction based on a Hartree-Fock reference state, and wherein the D₂ diagnostic is determined analogously to the D₁ diagnostic but refers to double excitations, and wherein the Zₛ₍₁₎ diagnostic is determined based on orbital-entanglement information obtained from an approximate correlated wavefunction such as a partially converged but qualitatively correct density matrix renormalization group (DMRG) wavefunction, and wherein the % TAE diagnostic is determined based on the difference of total atomization energies obtained with CCSD(T) and CCSD relative to the total atomization energy obtained with CCSD(T).

In a further aspect of the invention, a use of an apparatus as described above is presented for determining activation energies for a predetermined catalytic cycle and/or for determining reaction energies for a predetermined chelating agent.

In a further aspect, a control signal generated according to any of the apparatuses as described above, the methods as described above, and the computer program products as described above is presented.

It shall be understood that the apparatuses as described above, the methods as described above and the computer program products as described above have similar and/or identical preferred embodiments, in particular, as defined in the dependent claims.

It shall be understood that a preferred embodiment of the present invention can also be any combination of the dependent claims or above embodiments with the respective independent claim.

These and other aspects of the present invention will be apparent from and illustrated with reference to the embodiments described hereinafter.

### BRIEF DESCRIPTION OF THE DRAWINGS

In the following drawings:

FIG. 1 illustrates a state representation of a qubit as used in a quantum computing device,

FIG. 2 illustrates a schematic example of a quantum computing device with qubits as calculation unit,

FIG. 3 illustrates a schematic example method for generating a control signal to perform operations on a quantum computing device and for processing measurement signals from the quantum computing device,

FIG. 4 illustrates a schematic example of a hybrid system including a classical and a quantum computing device,

FIG. 5 illustrates a schematic example of a quantum computing device based on superconductors,

FIG. 6 illustrates a schematic example of a quantum computing device based on trapped ions,

FIG. 7 shows schematically and exemplarily an embodiment of a system for determining a solution of a problem,

FIG. 8 shows schematically and exemplarily a flow chart of a method for determining a solution of a problem,

FIGS. 9 to 12 show schematically and exemplarily flow charts of further details of the method for determining a solution of a problem for specific applications.

### DETAILED DESCRIPTION OF DRAWINGS

In the following first a short introduction into the general basic principles of quantum computers and the performance of calculations of quantum computers will be provided. Further, general principles can also be found in “Quantum Computation and Quantum Information: 10th Anniversary Edition”, M. A. Nielsen and I. L. Chuang (2010).

Classical computing devices use processors which are based on transistors. The state of each transistor has two controllable states 1 or 0 representing a digital binary or a bit. To perform operations on a classical computing device a human readable program code is translated via a compiler into machine-readable instructions. Machine-readable instructions are control signals, e.g. voltage settings, for each transistor. Representations of the machine-readable instructions may include binary or hexadecimal representations. Based on such machine-readable instructions, the operations are performed on the processor of a classical computing device.

Quantum computation is a relatively new computation method that uses quantum effects, such as superposition and entanglement, to perform certain computations more efficiently than classical digital computers. In contrast to digital computers, which represent information in the form of bits (e.g., “1” or “0”), as described above, quantum computing devices, i.e. quantum computers, use qubits, i.e. quantum bits, to represent information. Quantum computing devices are based on quantum elements adhering to the physics of quantum mechanics, such as superconductors, ions, atoms, quantum dots, photons, particle spins, bosons or the like. These quantum elements may be manipulated in a controlled manner to perform operations.

Although qubits and their manipulation may be described in terms of their mathematical properties, each such qubit may be implemented in a physical quantum element in any of a variety of different ways. Examples of such quantum elements include superconducting materials, trapped ions, photons, optical cavities, individual electrons trapped within quantum dots, point defects in solids (e.g., phosphorus donors in silicon or nitrogen-vacancy centers in diamond), molecules (e.g., alanine, vanadium complexes), or any medium that exhibits qubit behavior comprising quantum states and transitions there between that can be controllably induced or detected.

Generally, for any given physical quantum element that implements a qubit, any of a variety of properties of that physical unit may be chosen to implement the qubit. For example, if electrons are chosen to implement qubits, then the x, y or z component of an electron spin degree of freedom can be chosen as the property of such electrons to represent the states of such qubits. For any particular degree of freedom, the physical quantum elements can be controllably put in a state of superposition or entanglement and measurements can then be taken in the chosen degree of freedom to obtain readouts of qubit values.

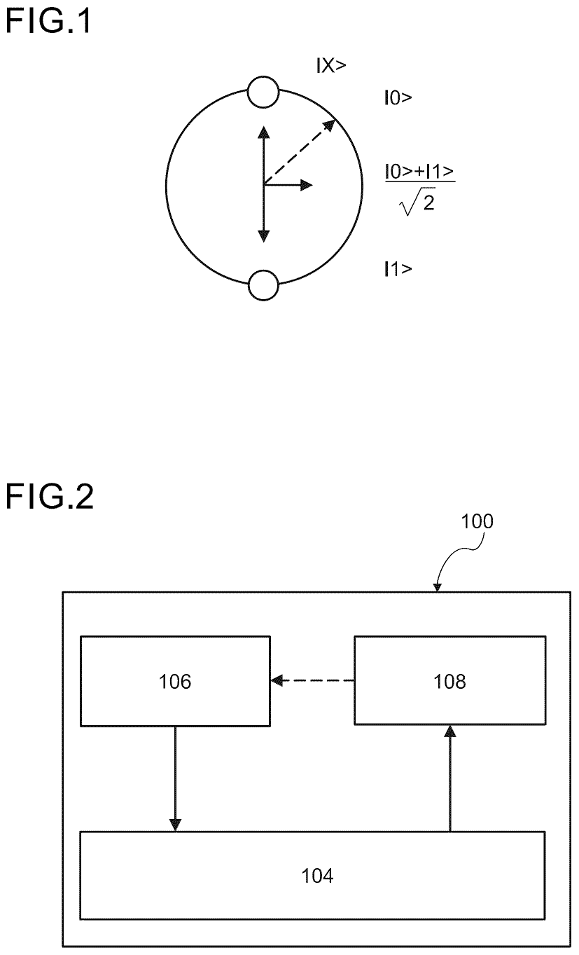

In contrast to transistors of classical computing devices each quantum element of quantum computing devices can not only take the basis states |1 [symbol] or |0 [symbol] but also any superposition of such basis states, such as state |X [symbol] . The state of each quantum element is represented by a state of a quantum bit, i.e. qubit, as illustrated in the two-dimensional simplification of FIG. 1. To represent such states Dirac notation is commonly used in quantum mechanics. In Dirac notation a state in a n dimensional, complex vector space, such as a Hilbert space, is represented in bracket notation, for example |X [symbol] . According to conventional terminology, the superposition of “0” and “1” states in a quantum computing device can be represented as α|0 [symbol] +β|1 [symbol] . The states “0” and “1” or bits of the classical computing device are similar to the basis states |0 [symbol] and |1 [symbol] or quantum bits of the quantum computing device, respectively. The value |α|² represents the probability that the qubit will be measured in the |0 [symbol] state, while the value |β|² represents the probability that the qubit will be measured in the |1 [symbol] state. If more than one qubit is present, two or more qubits may be entangled. Entanglement means that the state of one qubit is dependent on the state of at least one other qubit and vice versa, wherein further in the entangled state the respective qubits cannot be regarded as individual qubits anymore. Generally, a register of N qubits in a quantum computer can be put into a superposition of basis states at once whereas a register of N classical bits can only be in a single basis state at once. Thus, in contrast to classical computing devices on a quantum computing device 2^{N} basis states can be manipulated and processed simultaneously allowing for exponential intrinsic parallelism.

To perform operations on the quantum computing device the computational method to solve a given problem may be translated into qubit manipulations, which may be translated into control signals for manipulating qubits. Representations of the machine-readable instructions may include common quantum mechanical representations of operations in the Hilbert space. Depending on a specific realization of the quantum computer different representations of the qubit states may be chosen. Any state preparation on the quantum computing device may be represented by a manipulation acting on the qubit states. A manipulation may be translated into control signals to control a respective part of the quantum computer, which depend on the type of quantum computing device used. This way based on the manipulation acting on the qubit states, operations may be performed on the quantum equivalent of a classical processor as part of the quantum computing device.

In gate-based quantum computer systems the manipulations acting on the qubit states may generally be one- or multi-qubit operations. A one-qubit operation may change the state of one qubit e.g., into a specific superposition which corresponds to a rotation of the vector |X [symbol] as illustrated in FIG. 1. For example, in a superconducting quantum computer this can be accomplished by microwave pulses or in a trapped-ion quantum computer by irradiation of the ion with a laser beam. A multi-qubit operation may create entanglement between two or more qubits. For example, in a superconducting quantum computer this may be achieved by connecting qubits via an intermediate electrical coupling circuit or in a trapped-ion quantum computer via controlling the collective vibrations of the trapped ions.

Generally, to prepare manipulations for solving a given problem on a quantum computer a respective quantum mechanical representation of the problem may be translated into qubit manipulations, which are carried out to prepare a solution of the given problem. After the preparation of the predetermined solution, i.e. after the application of the operations to the qubits of the quantum computer, a projective measurement of all individual qubits is carried out returning either 0 or 1 for each qubit. This projection usually happens in the oz eigenbasis of the qubits which is also used to define the computational basis stats “0” and “1” of the qubit. This means that only operators that are products of oz operators or can be directly transformed into such operators can be measured concurrently. On the quantum computing device this measurement is achieved by applying a hardware-specific readout protocol of a series of readout manipulations including, for example, in the case of gate-based quantum computers, control pulses and monitoring the response to control pulses. For example, a superconducting qubit may be coupled to a hardware resonator. The measured shift of the resonator frequency allows to determine the state of the qubit as this shift depends on the state of the coupled qubit. In case of trapped ions, for example, an optical readout may be used, e.g. the state of the qubit is 1 if the ion emits light or 0 if the ion does not emit light or vice versa. This way qubits may be used, in particular, on gate-based quantum computers, to implement logical circuits or gates as in classical computing devices.

In FIG. 2 a schematic example of a quantum computer is illustrated. The quantum computing device 100 shown in FIG. 2 includes a quantum register 104 configured to perform the quantum computation, a manipulation part 106 configured to manipulate the quantum register, in particular, quantum elements forming the qubits, and a readout part 108 configured to collect measurement signals from the quantum register 104 for reading out the qubits after a quantum mechanical calculation. The manipulation part 106, in particular, provides manipulation signals for manipulating the quantum register, wherein the manipulation signals are generated based on received control signals that are determined based on the respective operations that should be performed on the qubits. In some embodiment a feedback loop between the manipulation part 106 and measurement part 108 can be provided. In contrast to classical computing, where one measurement cycle provides the state of a transistor, quantum computing includes performing multiple measurement cycles to provide a probability density or a probability for the qubit states in case of gate-based quantum computers.

The quantum register 104 can be based on different quantum elements representing the qubits. In some embodiments of gate-based quantum computers the qubits may be implemented by photons as quantum elements. Such optical quantum computing devices may include lasers that generate photons that are provided to a waveguide. A beam splitter can be provided for manipulating the photon states based on manipulation signals such as a mechanical rotation applied to a mirror. The measurement part 108 can in such an embodiment be a photon detector, and the measurement signals can be photons.

In other embodiments of gate-based quantum computers the qubits can be implemented by electronic states of ions trapped in a magnetic field. The manipulation part 106 can in such a case utilize a laser, and the manipulation signals can cause the providing of control laser pulses. Moreover, in this case, the readout part 108 can be a photon detector combined with read-out laser pulses, and the measurement signals 102 may be photons. Other qubit implementations may be based on superconductors as quantum elements, semiconducting material with anyons as quantum elements, or the like.

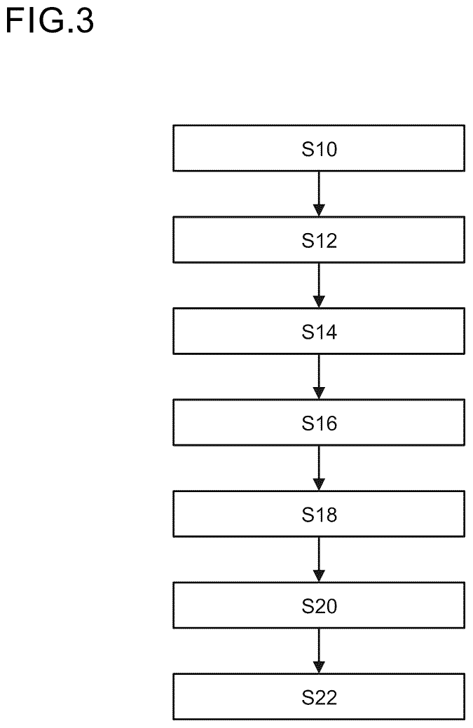

FIG. 3 illustrates a schematic exemplary method for generating a control signal to perform manipulations on a quantum computer and for processing measurement signals from the quantum computing device. In most embodiments of quantum computing devices known to date, the control signals for the quantum computing device are prepared on a classical computing device and the measurement signals provided by the quantum computing device are further processed on the classical computing device. Other embodiments are, however, conceivable as quantum computing devices mature. In the following example, the quantum computer refers to a gate-based quantum computer for which the manipulations refer to operations on the quantum elements of the quantum computer.

For generating the control signal to perform operations on the quantum computing device, the problem to be solved with the aid of the quantum computing device is provided in step S10, preferably, in a mathematical description. Such problems may for instance include determining a material property based on the mathematical description of the material's electronic structure. Other problems may include optimization problems and associated objective functions. Based on the problem to be solved, a quantum algorithm description of the problem or a sub-problem may be generated in step S12, wherein the quantum algorithm description comprises the operations to be applied to the qubits of the quantum computer to solve the problem in the quantum mechanical calculation. Further, the quantum algorithm description can include a reference state that allows to generate a representation of an initial qubit state on the quantum computer on which the further operations are then applied by manipulating the qubit states. Based on the quantum algorithm description control signals can then be generated in step S14 to control the quantum computer, for instance, by providing the control signals to a manipulation unit that can then manipulate the qubit states based on the control signals. In step S16 the manipulation unit then applies the manipulation operations to individual or multiple qubits of the quantum computer, wherein based on the manipulation operations the qubits perform the quantum mechanical calculation. After the manipulation, measurement signals can be generated to determine the result of the quantum mechanical calculation in step S18. This step can include a readout, i.e. measurement, of the qubit states after applying the manipulation operations to the initial qubit states. The measurement signals can in step S20 then be translated into a measured quantity on the classical computer and in case of a sub-problem fed back into the problem to be solved. Finally, the result of the problem calculation including the quantum mechanical calculation can be provided on the classical computing device in step S22.

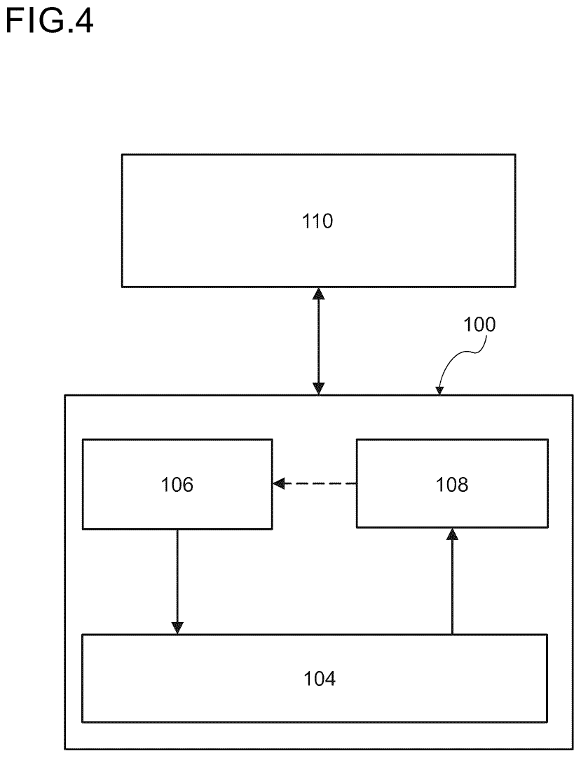

FIG. 4 illustrates a schematic example of a hybrid system including a classical and a quantum computing device. As described with respect to the method illustrated in FIG. 3, quantum computing devices are often used in connection with classical computing devices. As shown in FIG. 4 a problem preparation system, e.g. a control signal generation apparatus, can be realized as a classical computing device 110 performing, for instance, steps S10, S12, S20, S22 of the method illustrated in FIG. 3. A controlling unit can then be provided as interface between the classical computing device 110 and the quantum computer 100, wherein the controlling unit can also be a classical computing device, for instance, performing step S14. The control unit can then be communicatively coupled with the manipulation part 106 that can control the manipulators of the quantum computing device. Also, the manipulation part 106 can be realized as a classical computing device, for instance, a classical controlling hardware for the control of specific hardware components of the quantum computer that perform the manipulation of the qubits. However, the manipulation part 106 is generally regarded as part of the quantum computer, since it directly influences the quantum register. The quantum computing device 100 is adapted to perform the quantum operations in step S16, in particular, by the manipulation of the qubits of the quantum register. The measurement part 108 that is also generally regarded as part of the quantum computing device can then perform the step S18 by utilizing classical hardware. The measurement part 108 can then be communicatively coupled to the preparation system 110 for further processing of the measurement signals.

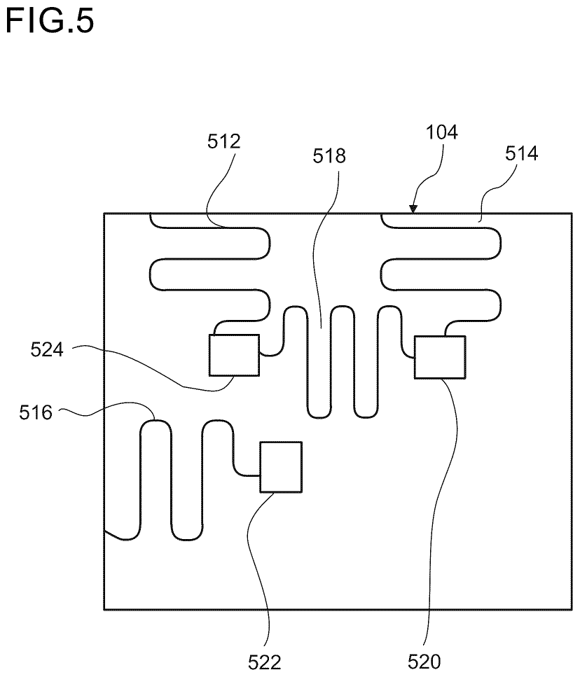

FIG. 5 illustrates a schematic example of a quantum computing device based on superconductors. Superconducting quantum computing devices are one of the solid-state quantum computing technologies. Here the quantum register 104 can include superconducting circuits 520, 522, 524 based on Josephson junctions. The qubits can then, for instance, refer to charge, flux, transmon, or phase qubits depending on the quantity of the superconducting circuits that are chosen to represent the qubits. FIG. 5 refers to a simplified illustration of a superconducting quantum computer utilizing charge qubits. For charge qubits the different states of the qubit are represented by an integer number of Cooper pairs on a superconducting island. In case of gate-based quantum computing quantum manipulations can then be performed by manipulating the qubits through microwave pulses. Resonators 512, 514, 516 can be utilized to manipulate the state of the qubits by applying the microwaves or for reading out the state of the qubits by measuring respective microwaves, wherein generally different resonators are used for the manipulation of the state of the qubits and the readout of the qubits. Moreover, resonator 518 can be utilized for applying microwaves that entangle the qubits. However, instead of resonator 518 the entanglement can also be achieved by an inductive or capacitive coupling of the superconducting circuits or even by providing another qubit, here a superconducting circuit, between the to be entangled qubits.

On an operational level such systems are maintained at extremely low temperatures, e.g., in the tens of mK. The extreme cooling of the systems keeps superconducting materials below their critical temperature and helps to avoid unwanted state transitions. To maintain such low temperatures, the quantum information processing systems may be operated within a cryostat, such as a dilution refrigerator. In some implementations, control signals are generated in higher-temperature environments, and are transmitted to the quantum computer using shielded impedance-controlled GHz capable transmission lines, such as coaxial cables. In some implementations, the state measurement of superconducting qubits is achieved using a dispersive detection scheme. In order to read out or detect the state of any qubit, a probing signal, e.g., a travelling microwave, may be excited along a readout transmission line coupled to the qubit via a respective readout resonator. The frequency of the probing signal can be in the vicinity of the resonance frequency of the readout resonator. Depending on the internal quantum mechanical state of the qubit, the intensity or phase of the probing signal transmitted along the readout transmission line may be altered because the reflectivity of the readout resonator coupled to the qubit changes depending on the state of the qubit. This allows for the state detection of the qubits, wherein during the readout of a qubit state the state of the qubit collapses, i.e. is projected with the respective probability onto one of the basis states. By performing the quantum mechanical calculation and the readout a plurality of times, for example, respective probabilities can be determined. Further details for superconducting quantum devices are described e.g. in documents EP 3830867 A1, EP 3449427 A1, US2020272925 A1, CN 212061223 U and U.S. Pat. No. 2019019099 A1.

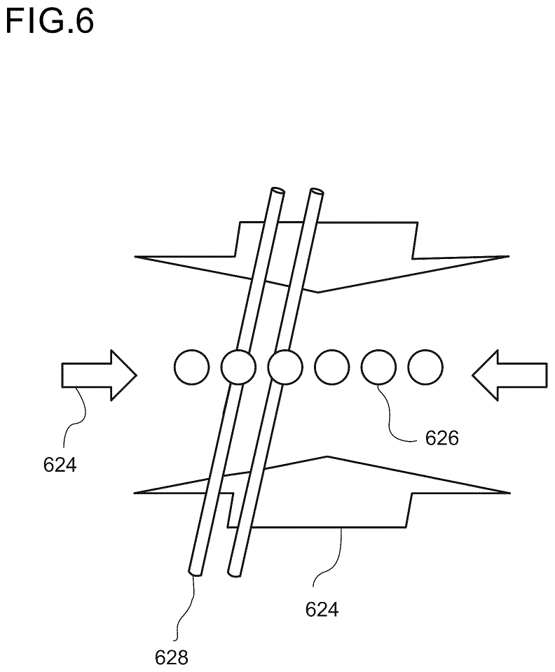

FIG. 6 illustrates a schematic example of a quantum computing device based on ions in an ion trap. Similar to neutral atom traps ion traps with, e.g. positively charged Calcium ions, can be used to implement the quantum computing device. Here ions 626 are trapped in an oscillating electromagnetic field 624 inside a high or ultra-high vacuum. The ions 626 are laser cooled and held in the oscillating electrical field 624. For qubit manipulation such as superposition or entanglement laser light 628 at different frequencies may be used.

Generally, based on the above described quantum computer realizations gate-based type calculations can be performed on a quantum computer hardware architecture. The gate-based type calculation is based on quantum gates. In contrast to classical gates, there is an infinite number of possible single-qubit quantum gates that can change the state vector of a qubit. Changing the state of a qubit state vector typically is referred to as a single qubit rotation, and may also be referred to herein as a state change or a single-qubit quantum gate operation. A rotation, state change, or single-qubit quantum gate operation can be represented mathematically by a unitary 2×2 matrix with complex elements. A rotation corresponds to a rotation of a qubit state within its Hilbert space, which can be conceptualized as a rotation of a vector on the Bloch sphere, wherein the Bloch sphere is generally known as a geometrical representation of the space of the pure states of a qubit. Multi-qubit gates alter the quantum state of a set of qubits. For example, two-qubit gates rotate the state of two qubits as a rotation in the four-dimensional Hilbert space of the two qubits, wherein, as generally known, the Hilbert space is an abstract vector space possessing the structure of an inner product that allows length and angle to be measured. Furthermore, Hilbert spaces are complete, i.e. there are enough limits in the space to allow the techniques of calculus to be used.

In the following the term quantum algorithm description refers to a representation of a problem that comprises either a sequence of quantum operations that should be applied during a quantum mechanical calculation of the problem on a gate-based quantum computer. The term “quantum manipulation” can include in the context of this invention all types of quantum gates as described above and more generally all manipulations known for quantum elements on any quantum computer hardware. Further, in some applications the quantum manipulations can also include measurement manipulations. This allows to implement algorithms using a measurement feedback. For example, in such an algorithm a quantum computer can execute the quantum gates defined by the sequence of quantum manipulations and then measure only a subset, i.e., fewer than all, of the qubits or other calculation elements, like the bosonic field states, in the quantum computer, and then decide which further quantum manipulations to execute next based on the outcome of the one or more measurements. In particular, measurement feedback can be useful for performing quantum error correction, but is not limited to use in performing quantum error correction.

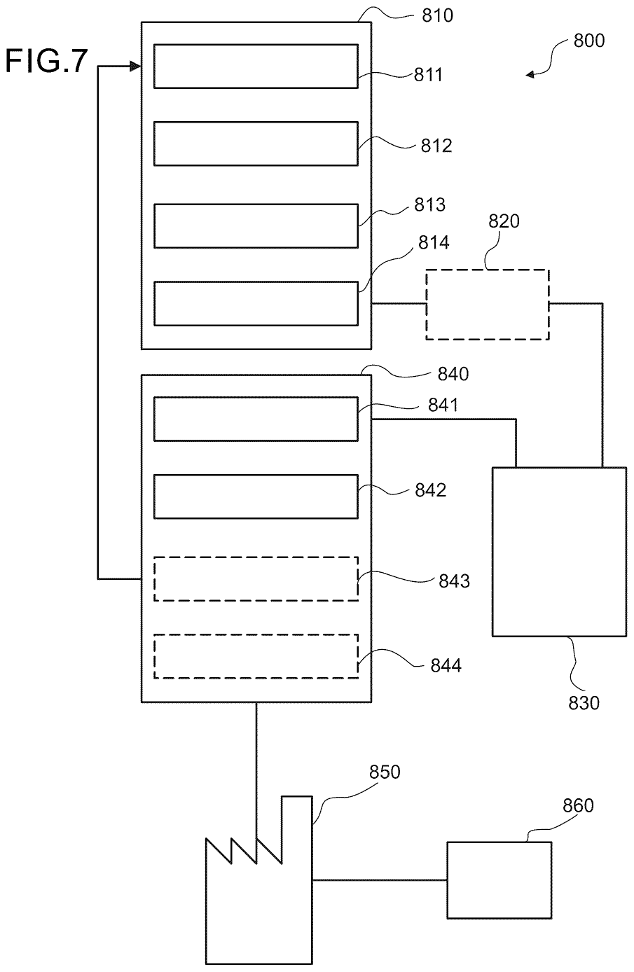

FIG. 7 shows schematically and exemplarily a system for determining a solution of a problem. In particular, the example shown in FIG. 7 refers to determining the solution of a problem that allows to determine a target chemical product comprising a target technical application property. However, in other examples the problem can also refer to other applications related to chemical products, solid product or even to problems that are completely unrelated to chemical products, for instance, to scheduling problems, cryptographic problems, etc. The system 800 comprises in this exemplary embodiment an apparatus 810 for generating control signals for measuring states of quantum elements of a quantum computer, an apparatus for determining a solution of a problem 840, and optionally also a quantum computing system 830. Generally, the control signal generation apparatus 810 and the solution determination apparatus 840 are communicatively coupled to the quantum computer 830. The quantum computer 830 can refer to any quantum computer as described above, for example, can refer to a gate-based quantum computer. Further, the solution determination apparatus 840 can optionally be communicatively coupled with a production system 850, in particular, with the production management system adapted to manage and control a production of the production system 850 to provide control signals to the production system that initiate the production of one or more chemical products 860.

The control signal generation apparatus 810 is configured for generating control signals for measuring states of quantum elements of a quantum computer. In particular, the control signal generation apparatus 810 comprises a problem providing unit 811, a transformation unit 812, a translation unit 813 and a control signal generation unit 814. Generally, the control signal generation apparatus can be realized as any known classical computing system. For example, the function provided by the units can be performed by one or more processors on one or more classical computing devices. In particular, the control signal generation apparatus 810 can also be realized as a distributed computing framework, for instance, a cloud or network environment, in which more than one computing device is utilized for performing the functions of the apparatus.

The problem providing unit 811 is configured for providing a problem description indicative of the problem to be solved. For example, the problem providing unit 811 can be communicatively coupled with a user interface to allow a user to indicate, for instance, select from a respective provided selection, the respective problem to be solved. Generally, the problem description is translatable into a quantum mechanical description in order to be solvable by a quantum computer. In the exemplary embodiment shown in FIG. 7, a solution of the problem is indicative of a technical application property of a chemical product and can thus be utilized for determining this technical application property. Generally, the problem description can be provided in any form that allows the apparatus 810 to derive respective information from the problem description, in particular, the problem description can be provided in a digital format. Preferably, the problem description refers to a mathematical description of the problem, for example, to a mathematical representation of an electronic structure problem, or a mathematical representation of an optimization problem. However, the problem description can also be provided in any other format from which the problem providing unit 811 or optionally also the transformation unit 812 can derive a respective mathematical formulation. The problem providing unit 811 can then provide the problem description to the transformation unit 812.

The transformation unit 812 is configured for transforming the problem description into a quantum mechanical representation comprising one or more operators representing one or more observables to be measured indicative of the solution of the problem. In particular, the quantum mechanical representation refers to a representation of the problem that represents a state of the quantum mechanical system to be prepared on the quantum computer 830 such that the respectively prepared states of the quantum elements of the quantum computer 830 are representative of the operators that are indicative of the solution of the problem. Methods and algorithms for transforming a problem that is translatable into a quantum mechanical description into such a quantum mechanical representation that can be prepared on a quantum computer for solving the problem are well-known and some of them will be described later in more detail. Further, the transformation unit is configured such that the transformation additionally comprises determining unitary transformations that rotate the one or more operators representing one or more observables to be measured into one or more bases that lead to a pure occupation number representation of the one or more operators after the application of the unitary transformations. In particular, the transformation unit determines which unitary transformations are necessary for transforming the quantum mechanical representation into a pure occupation number representation. For determining the unitary transformations depending on the specifics of the problem and the quantum mechanical representation of the problem, a plurality of different methods and algorithms can be utilized that will later be described in more detail with respect to the respective application cases.

The translation unit 813 is then configured to translate the quantum mechanical representation into a quantum algorithm description comprising a sequence of quantum operations to be applied to the quantum elements of a quantum computer. Generally, the quantum algorithm description comprises the quantum operations that are necessary to prepare the quantum mechanical representation of the problem on the quantum computer and further to measure the states of the quantum elements representing the observables of the quantum mechanical representation and thus a solution of the problem. Respective translation algorithms that determine a quantum algorithm description based on a respective quantum mechanical representation are known and can be utilized by the translation unit. The sequence of quantum operations, i.e. the quantum algorithm description, comprises a preparation part and a measuring part. The preparation part generally comprises the quantum operations that can be utilized for preparing the quantum mechanical representation of the problem on the quantum computer such that the observables being indicative of the solution of the problem are measurable. The measuring part of the quantum algorithm description then refers to the quantum operations that are applied to the quantum elements after the preparation of the quantum mechanical representation on the quantum elements in order to measure the respective states of the quantum elements. In particular, in this invention, the measurement operations comprise unitary transformation operations to be applied to the quantum elements. The unitary transformation operations are in particular determined by the translation unit by utilizing the unitary transformations determined by the transformation unit. In particular, the unitary transformation operations are determined such that the unitary transformation operations initiate a rotation of the states of the quantum elements into respective basis states corresponding to the one or more bases leading to the pure occupation number representation of the one or more operators representing the one or more observables to be measured. Thus, the determined unitary transformation operations initiate a basis transformation of the states of the quantum elements, wherein a value of the observables is not changed by this basis transformation. Accordingly, although the states of the quantum elements in the new bases differ from the states of the quantum elements before the basis transformation, the states of the quantum elements still are indicative of, in particular, represent, the observables and thus the solution of the problem. Applying the unitary transformation operations such that the operators representing the one or more observables refer to a pure occupation number representation allows to measure the states of the respective quantum elements concurrently, during the same preparation of the quantum mechanical representation, whereas otherwise for measuring the states of the quantum elements the quantum mechanical representation has to be prepared anew for measuring a state of each quantum element representing an observable that is not commutable and has no compatible basis in the quantum elements. Thus, respective preparations of the quantum mechanical representation and respective measurements can be saved allowing either to minimize the quantum computational resources necessary for performing a quantum computer calculation, or to utilize the freed resources to calculate more complex problems, i.e. problems with more variables, or to determine the problems more accurately, for instance, by utilizing a respective better statistic or by utilizing respective error correction methods that would otherwise need too many resources.

The control signal generation unit 814 is then configured to generate control signals for controlling the application of the determined sequence of quantum operations, i.e. the quantum algorithm description, to the quantum computer such that the quantum mechanical representation of the problem is prepared and the observables indicative of the solution of the problem are measured. In particular, the control signals can refer to any format that can be read by the quantum computer 830 or an optional quantum computer management system 820. In particular, the control signals can be generated such that they directly allow for a controlling of the quantum computer 830, in particular, of manipulation units or parts of the quantum computer 830 for manipulating the quantum elements in accordance with the determined quantum algorithm description. However, in most cases the quantum computer 830 will be provided with a quantum computer management system 820 that is configured for the direct controlling of the quantum computer 830 and in most cases further, for example, for the scheduling of respective calculations, etc. In such cases, the control signals can be generated such that they are readable and interpretable by the quantum computer management system 820 such that the quantum computer management system 820 controls the quantum computer 830 in accordance with the determined quantum algorithm description.

The quantum computer 830 can then provide the results of the measurement of the observables to the solution determination apparatus 840. In particular, a quantum computer interface unit 841 can be utilized to receive the respective results of the measurements of the observables that can be provided in any format that is readable by the apparatus 840. Generally, the apparatus 810 and the apparatus 840 can together form a solution apparatus but can also be provided as independent units. In particular, the solution determination apparatus 840 can be performed by the same hard- and/or software as the control signal generation apparatus 810, for instance, can be realized on the same computing device or by the same computer network as part of a respective solution framework. However, the solution determination apparatus 840 can also be provided independent of the control signal generation apparatus 810, for instance, can be provided on a different hard- and/or software, for example, on a different computing device.

A determination unit 842 of the solution determination apparatus 840 is then configured to determine a solution of the problem based on the received measurement results from the quantum computer. For example, any known method and/or algorithm can be utilized that allows to determine based on the measurement results of states of quantum elements the solution of the respective problem. In particular, known mathematical formulations can be used to determine values for the respective observables based on the measurements results. These values for the respective observables can then be utilized to determine a solution of the problem. Since in this example the problem refers to the determination of a technical application property, the respective determined solution of the problem, for example, an energy of a molecule, can then be utilized by an optional property determination unit 843 to determine a technical application property of the chemical product based on the determined solution of the problem.

Optionally, an iteration unit 844 can be provided that compares the determined technical application property of the potential chemical product with the target technical application property, i.e. with the target value of a target technical application property. If the determined technical application property meets the target technical application property within predetermined limits, i.e. if the determined technical application property fulfils a predetermined criterion, the chemical product can be determined as the target chemical product. If the comparison indicates that the determined technical application property does not meet the target technical application property within the predetermined limits, a new potential chemical product can be provided and the determination of the technical application property can be repeated utilizing the new potential chemical product. The new potential chemical product can be provided, for example, by amending one or more constituents or synthesis parameters of the previous chemical product in order to allow to generate a new potential chemical product with different characteristics. For example, if the chemical product refers to a polymer, a respective synthesis specification of a previous polymer can be arbitrarily or according to predetermined rules amended to generate a new synthesis specification referring to a new polymer. Generally, this process can be performed without synthesizing or producing the chemical product until a final target chemical product is determined. The providing of the new chemical product can thus be an automatic process that can also be a supervised process in which a user is presented with potential new chemical products and selects one or more of the new chemical products for the further processing, or the new chemical product can be provided by the user. Moreover, the new potential chemical product can also be determined by selecting the new potential chemical product from a list of previously determined potential chemical products. Based on the new potential chemical product again, for example, a respective problem can be provided. In particular, in most cases the new potential chemical product will not lead to a change of the type of a problem to be solved but will, for example, change one or more constants, variables or other quantities of the problem such that the process as described above can be performed based on this new problem referring to the new potential chemical product. If at the end of the process finally a target chemical product is determined, control data comprising, for instance, a recipe and/or constituents of a target chemical product, can be provided to production system 850 and based on this control data the target chemical product 860 can be produced.

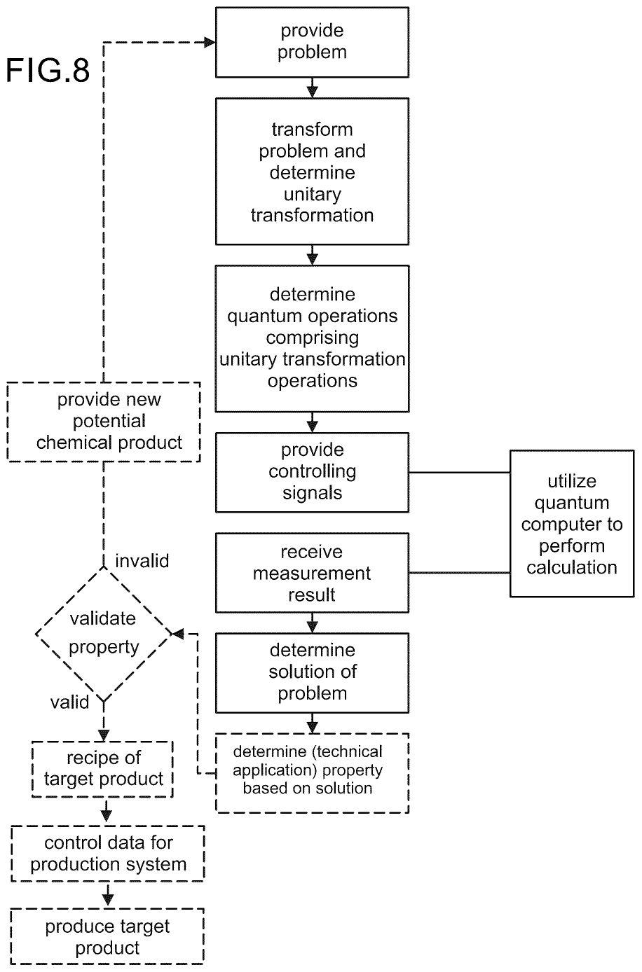

FIG. 8 shows schematically and exemplarily a flowchart of a method for determining a solution of a problem utilizing a quantum computer according to the invention. In particular, the method can be performed by utilizing the system as described above with respect to FIG. 7. In a first step, the method comprises providing a problem description indicative of the problem to be solved. For example, corresponding to the example provided in FIG. 7, the problem relates to the determination of a technical application property of a chemical product. In a next step, the problem description is transformed into a quantum mechanical representation. Further, unitary transformations can be determined that rotate operators of the quantum mechanical representation into bases that lead to a pure occupation number representation of the operators. Generally, the explanations and principles already described above with respect to the apparatus, in particular, with respect to the transformation unit, can be applied in this step. In a next step, a quantum algorithm description is determined based on the quantum mechanical representation and based on the unitary transformations such that the quantum algorithm description comprises unitary transformation operations that initiate a rotation of states of the quantum elements of a quantum computer into respective basis states corresponding to the bases leading to the pure occupation number representation of the one or more operators. Also in this step the principles already described above with respect to FIG. 7, in particular, with respect to the translation unit, can be applied. Based on the such determined quantum algorithm description, control signals can be generated that allow for a controlling of a quantum computer in order to perform the quantum algorithm description for preparing the quantum mechanical representation on the quantum computer and measuring the states of the respective quantum elements. Thus, based on the control signals the quantum computer can then be utilized to perform the quantum computer calculation and for providing the results of the measurements of the states of the quantum elements.

The respective measurement results can then be received, for example, by an apparatus like apparatus 840 and utilized to determine a solution of the problem. Optionally, the method can further comprise in this example determining a property of a chemical product based on the determined solution. The respective technical application property can then be validated, for example, the respective technical application property can be validated against one or more predetermined criterions like a target technical application value. If the validation indicates that the technical application property fulfils the respective criterions, i.e. is “valid”, the respective chemical product can be determined as the target chemical product. However, if the validation indicates that the determined technical application property does not fulfil the predetermined criterions, i.e. is “invalid”, a new potential chemical product can be provided. Based on the new potential chemical product, a corresponding new problem can be determined and the respective process can be repeated. If a potential chemical product is determined as target chemical product, control data can be generated for controlling a production system to produce the chemical product. Finally, the method can then comprise producing the target chemical product based on the provided control data.

In the following, preferred embodiments of the invention will be described in detail with respect to preferred application cases. Generally, quantum computing is an emerging technology that exploits quantum-mechanical phenomena to perform computational tasks. Quantum computers are expected to solve certain computational problems substantially faster than classical computers. They can be used for example to simulate quantum-mechanical systems, such as electronic structure systems, including but not limited to molecules, crystals and amorphous solids. Additionally, optimization problems, machine learning and artificial intelligence are further exemplary application fields of quantum computing. Quantum computing is expected to significantly surpass classical computing with regard to treatable problem size, required computation time and/or achievable accuracy in the abovementioned fields.

The fundamental processing unit of a quantum computer is a quantum-mechanical bit (qubit). By executing a suitable quantum circuit, e.g. based on a quantum algorithm description, via control pulses that act on the qubits, a solution to one of the abovementioned problems or a specific subproblem is prepared in the qubit register. To read out this solution and provide the result to the user, each qubit is measured by applying control pulses followed by a hardware-specific readout protocol that projects the state of each qubit onto one of its two energy eigenstates, i.e. providing the eigenvalues 0 or 1. The control pulses are used, e.g., to rotate the state of the qubit into a basis representing the solution in the measurement basis of the qubit. This quantum-mechanical measurement is inherently a statistical process and to obtain a meaningful result one needs to average over many repeated state preparations via the aforementioned quantum circuit and measurements. The number of required measurements typically depends on the size of the problem under study, the observable to be measured and thereby the number of elementary operators representing the observable to be measured, the desired accuracy and further algorithm specifics. Especially in case of hybrid quantum-classical computing, which combines quantum computing with classical computing and is particularly well-suited to leverage the limited capabilities of near-term noisy intermediate-scale quantum (NISQ) computers, one of the major obstacles is typically the large number of required measurements.

In quantum computing the information about the system under study can be extracted by measuring observables. Generally, observables are representable by operators in a respective quantum mechanical formulation, thus in the following the terms operator and observable can be utilized interchangeably keeping in mind this relation. In general, an observable is given by a Hermitian operator Ô that can be written as a sum of up to D Hermitian tensors V, with dimensionality of up to 2D

$$\hat{O} = {\sum }_{d}^{D} {\hat{V}}_{d} with {\hat{V}}_{d} = {\sum }_{{p}_{1} , … , {p}_{d} , {q}_{1} , … , {q}_{d}}^{N} {V}_{{q}_{1} … {q}_{d}}^{{p}_{1} … {p}_{d}} {\hat{c}}_{{p}_{1}}^{†} {\hat{c}}_{{q}_{1}} … {\hat{c}}_{{p}_{d}}^{†} {\hat{c}}_{{q}_{d}} \qquad ( 1 )$$

with V and D depending on the specific quantity of interest. In case an electronic structure problem is considered

$${\hat{c}}_{{p}_{i}}^{†} and {\hat{c}}_{{p}_{i}}$$

represents the electronic creation and annihilation operators, respectively. The indices p₁, . . . , p_{d}, q₁, . . . , q_{d} are running from 1 to N with N being proportional to the size of the system that is to be treated on the quantum computer. As can be easily seen, the number of individual terms contained in Ô is

$${N}_{terms} ∝ {N}^{2 D} \qquad ( 2 )$$

To obtain the measurement result, we need to determine the quantum-mechanical expectation value

$$〈 \hat{O} 〉 = 〈 Ψ \vert \hat{O} \vert Ψ 〉 \qquad ( 3 )$$

of the operator Ô with respect to the quantum state |ψ [symbol] prepared on the quantum computer by means of a quantum circuit, e.g. quantum algorithm description. For this typically the operator averaging technique is used, i.e., each of the Nₜₑᵣₘₛ individual terms is rewritten into sums over products of Pauli operators and measured individually.

Since the expectation value in Eq. (3) is determined stochastically the projective measurement on the quantum computer has to be repeated for each term requiring in total an order of

$$𝒪 ( {N}_{terms}^{} / {ϵ}^{2} )$$

state preparations anu measurements to obtain the expectation value with accuracy ε. Thus, the total number of measurements (for each measurement the state needs to be prepared once) is

$${N}_{meas} ∝ {N}^{2 D} / {ϵ}^{2} \qquad ( 4 )$$

which clearly becomes infeasible as N and/or D becomes large.

In the following, as an important example, the Hermitian operator Ô refers to a general Hamiltonian Ĥ that refers to the energies of electronic structure systems like molecules, solids and materials. This Hamiltonian has the general form:

$$\hat{H} = {\sum }_{pq}^{N} {h}_{pq}^{′} {\hat{c}}_{p}^{†} {\hat{c}}_{q} + \frac{1}{2} {\sum }_{pqrs}^{N} {v}_{pqrs} {\hat{c}}_{p}^{†} {\hat{c}}_{q} {\hat{c}}_{r}^{†} {\hat{c}}_{s} \qquad ( 5 )$$

In the most general case of a relativistic Hamiltonian, h′ and v are 2- and 4-dimensional complex tensors, respectively (i.e. D=2). v contains the two-electron integrals, which are expressed in Dirac notation as

$${v}_{pqrs} = 〈 pr \vert \hat{v} \vert qs 〉 \cdot {h}_{pq}^{′} = {h}_{pq} - \frac{1}{2} {\sum }_{r} {v}_{prrq}$$

contains one-electron integrals h_{pq}= [symbol] p|ĥ|q [symbol] along with an effective one-electron contribution originating from two-electron integrals. While h is Hermitian, v is symmetric with respect to the interchange of double indices pq and rs, and it is Hermitian with respect to the simultaneous interchange of p with q and r with s:

$${v}_{pqrs} = {v}_{rspq} = {v}_{qpsr}^{*} = {v}_{srqp}^{*} .$$

The term representing the repulsion of the nuclei is not shown in Eq. (5) as it is simply a constant within the Born-Oppenheimer approximation.

In case of a fully relativistic Hamiltonian, p, q, r, s are running over N four-component complex spinors

$${ϕ}_{p} = {( {ϕ}_{p}^{L , ↑} , {ϕ}_{p}^{L , ↓} , {ϕ}_{p}^{S , ↑} , {ϕ}_{p}^{S , ↓} )}^{T}$$

describing molecular orbitals (MOS). The first two entries are called the large component and describe spin-up and spin-down electrons. The last two entries are called the small component and describe spin-up and spin-down positrons. As relevant energies in typical electronic structure calculations are well below the electronic rest energy, electron-positron pair creation and annihilation can be neglected and a restriction to the subspace of electronic states typically is a very good approximation. This so-called “no pair” approximation is also a pre-requisite for the formulation of stable and robust relativistic electronic structure methods.

The Hermitian one-electron integral terms h_{pq} are calculated via the Hermitian Dirac operator

$$\hat{h} = ( \begin{matrix}{h}_{eN} {σ}^{0} & c ( \vec{σ} \cdot \hat{\vec{p}} ) \\ c ( \vec{σ} \cdot \hat{\vec{p}} ) & ( {h}_{eN} - 2 {c}^{2} ) {σ}^{0}\end{matrix} ) \qquad ( 6 )$$

which is a 4×4 matrix. c is the speed of light, \vec{σ}=(σ^{x}, σ^{y}, σ^{z})^{T} the vector of 2×2 Pauli spin matrices, σ⁰ the 2×2 identity matrix, \vec{\hat{p}} the general momentum operator and h_{eN} the electron nucleus interaction potential. Additional electrical potentials can be absorbed in h_{eN}.

In case of a fully relativistic Hamiltonian the interelectronic interaction is described by the Coulomb-Breit operator that includes all possible interaction terms up to [symbol] (1/c²) in quantum electrodynamics. The complex two-electron integral terms v_{pqrs} are then obtained via the Hermitian operator

$$\hat{v} = \frac{1}{{r}_{12}} - \frac{1}{2 {r}_{12}} ( {\vec{α}}_{1} \cdot {\vec{α}}_{2} + \frac{( {\vec{α}}_{1} \cdot {\vec{r}}_{12} ) ( {\vec{α}}_{2} \cdot {\vec{r}}_{12} )}{{r}_{12}^{2}} ) \qquad ( 7 )$$

with \vec{r}₁₂=\vec{r}₁−\vec{r}₂ and r₁₂=|\vec{r}₁₂|. \vec{α} is the vector of the traceless 4×4 matrices

$${α}^{x , y , z} = ( \begin{matrix}0 & {σ}^{x , y , z} \\ {σ}^{x , y , z} & 0\end{matrix} ) .$$

Neglecting the magnetic contributions to the interelectronic interaction as well as the gauge term recovers the ordinary Coulomb operator (first term) in Eq. (7), which represents the non-relativistic limit of the interelectronic interaction.

To simplify the complexity of the underlying equations and speed up the required computation time for integral evaluation, a reduction to a “quasi-relativistic” purely electronic two-component Hamiltonian can be carried out that also includes the most important relativistic effects to a good approximation. To this end the electronic degrees of freedom (large component) can be completely decoupled from the positronic degrees of freedom (small component) arriving at the two-component complex spinors

$${ϕ}_{p} = {( {ϕ}_{p}^{L , ↑} , {ϕ}_{p}^{L , ↓} )}^{T}$$

and the Hermitian 2×2 one-electron operator

$$\hat{h} = ( \frac{{\hat{\vec{p}}}^{2}}{2} + {h}_{e N} + {\hat{h}}_{s r} ) 1 + \vec{σ} \cdot {\hat{\vec{h}}}_{s o} \qquad ( 8 )$$

that is used instead of Eq. (6). ĥₛᵣ is the scalar relativistic contribution that contains the mass-velocity term as well as the Darwin term. The non-diagonal contribution \vec{σ}·\vec{ĥ}ₛₒ describes the spin-orbit operator. There are several well-established procedures to efficiently carry out the reduction from a fully relativistic four-component Hamiltonian to a “quasi-relativistic” two-component Hamiltonian and arrive at expressions for h that are of the structure that is given by Eq. (8), e.g. effective core potentials (ECPs), Douglas-Kroll-Hess (DKH) Hamiltonians, Barysz-Sadlej-Snijders (BSS) Hamiltonians, infinite-order two-component (IOTC) Hamiltonians, exact quasi-relativistic (XQR) Hamiltonians, the theory of exact two-component decoupling (X2C) or the zeroth order regular approximation (ZORA).

In case of a “quasi-relativistic” Hamiltonian the interelectronic interaction from Eq. (7) reduces to its non-relativistic limit, the Coulomb operator

$$\hat{v} = \frac{1}{{r}_{12}} \qquad ( 9 )$$

In case the spin-orbit operator in Eq. (8) is neglected, the ordinary non- or scalar-relativistic Hamiltonian is obtained. In that case the spinors can be chosen as real-valued eigenfunctions of the ŝ_{z} spin operator, i.e. as spin-up and spin-down molecular orbitals,

$${ϕ}_{p ↑} = {( {ϕ}_{p}^{L , ↑} , 0 )}^{T} and {ϕ}_{p ↓} = {( 0 , {ϕ}_{p}^{L , ↓} )}^{T} ,$$

respectively. With this h and v in Eq. (1) can both be chosen as real and symmetric tensors.

Measuring the expectation value of the (relativistic) Hamiltonian with respect to the prepared quantum state is of significant importance since it yields the energy spectrum of the electronic structure system such as a molecule or solid. This plays a crucial role within variational hybrid quantum-classical approaches where the iterative procedure is repeated until the lowest possible energy, e.g., the ground state, is reached. Furthermore, the obtained energy allows for the prediction of chemical reactivity, e.g., thermodynamics and kinetics, as well as spectroscopic properties of electronic structure systems. Since the number of terms in all Hamiltonians described above scales as Nₜₑᵣₘₛ∝N⁴ and thus the number of required non-concurrent measurements in the worst case scales as Nₘₑₐₛ∝N⁴/ϵ², such calculations become infeasible for larger system sizes.

In cases where the exact solution of the full electronic structure problem cannot be prepared on the quantum computer it might be necessary to enhance the overall accuracy in a post-processing step which then typically requires the measurement of even higher-dimensional terms (i.e. D>2). This represents an even more severe measurement bottleneck as the general worst-case scaling for the number of required non-concurrent measurements is Nₘₑₐₛ∝N^{2D}/ϵ².

However, some methods have been proposed for decreasing the number of measurements. For example, a simple but not general strategy is to identify terms (e.g. h_{pq} and v_{pqrs} in case of an electronicstructure Hamiltonian) that are below a certain cut-off threshold. These terms can then be neglected to reduce the number of required measurements. Moreover, by grouping of terms the measurement of several products of Pauli operators can be combined. With this the number of measurements can be reduced by a constant factor. Furthermore, a method to reduce the number of measurements that utilizes constraints originating from the problem structure has been proposed. It has been shown for an exemplary electronic structure problem that n-representability constraints can be used to reduce the number of required measurements by around one order of magnitude. Furthermore, with a local set of molecular orbitals and in the asymptotic limit of very large systems, the required number of measurements reduces to Nₘₑₐₛ∝N²/ϵ². When employing the Hubbard model Hamiltonian, the number of measurements reduces to Nₘₑₐₛ∝1/ϵ². Another method to reduce the number of required measurements is the classical shadow approach that in principle allows for a logarithmic dependence on the number of individual terms. However, this approach is ill-suited for hybrid approaches in electronic structure theory.

To make quantum computing generally usable for solving problems being translatable into a quantum mechanical description, in particular for solving such problems that refer to large systems, i.e. large N, and/or refer to observables to be measured that are represented by operators that are connected to higher-dimensional tensors, i.e. large D, solutions need to be found to reduce the number of required measurements by preparing the measurement process on a classical computer, controlling the respective quantum computer measurement signals and assembling the results from the quantum computer on a classical computer to obtain the expectation value [symbol] Ô [symbol] for a general Hermitian problem operator Ô. Moreover, it would also be advantageous if the respective solution could be applied to a wide variety of quantum mechanical problems, in particular, also problems utilizing a relativistic formulation.

In this context the invention utilizes the fact that terms that have compatible control pulse sequences, e.g., because different qubits are measured or a qubit is measured in the same basis for different terms, can be measured within a single state preparation. This can reduce the total number of state preparations and measurements by a system-dependent factor. An example for such a case is a Hamiltonian with only pure density-density interactions. For such a case all terms can be measured concurrently and the number of state preparations reduces to an order of [symbol] (1/ϵ²). For a generic system as discussed above this method does not change the overall scaling.

Accordingly, the solution to the above problem according to the invention is based on this principle. In particular, a low-rank decomposition can be used to improve the measurement process relevant for (relativistic) electronic structure calculations on a quantum computer. Preferably, this method refers to a hybrid quantum-classical procedure that improves the measurement process by reducing the number of non-concurrent measurements on the quantum computer, Nₘₑₐₛ, from [symbol] (N^{2D}/ϵ²) to [symbol] (L/ϵ²) with L being the dimension of a set of rotated bases, e.g. the number of auxiliary basis functions in the case of a factorization via the resolution of the identity (RI) approximation. Examples are general one-electron operators (i.e. D=1) and the two-electron operators connected to v in the (relativistic) electronic structure Hamiltonian (i.e. D=2). For measuring those, Nₘₑₐₛ can be reduced from [symbol] (N²/ϵ²) to [symbol] (1/ϵ²) and [symbol] (N⁴/ϵ²) to [symbol] (N/ϵ²), respectively. Whereas in the case of one-electron operators no auxiliary functions are required, in the case of the abovementioned two-electron operators the scaling can be improved by utilizing an appropriate set of auxiliary functions. These improvements overcome one of the major obstacles in quantum computing for (relativistic) electronic structure problems, namely the large number of required measurements. They thus allow for simulation of larger problems, e.g. larger molecules, more efficient determination of their energy spectrum via improved measurement of the (relativistic) Hamiltonian from Eq. (1) as well as more efficient determination of physicochemical properties which are typically connected to one-electron operators.

Moreover, utilizing a resolution of the identity (RI) approximation for an improved measurement of the two-electron terms in a Hamilton operator formulation of a problem leads to the number of rotated bases equaling the number of auxiliary basis functions utilized. This leads to the advantages described in the following. In particular, the number of rotated bases increases in this case proportionally with the number of auxiliary basis functions and thus also proportionally with the size of the electronic structure problem, for instance, the size of a molecule or the number of orbitals in a molecule. Without the RI approximation the number of rotated bases increases normally in quadratic or optimally in linear relationship with the size of the electronic structure problem. Moreover, if in this case the relationship is already asymptotically linear, utilizing a RI approximation leads to even less rotated bases while the error introduced by the approximation typically is negligible. Thus, for measuring the observables less resources are needed. Moreover, when utilizing the RI approximation it is not necessary to perform a factorization of two-electron integrals on a classical computer when preparing the problem for the quantum computer. Since the factorization can be computationally expensive depending on the problem, in particular depending on the size of the electronic structure problem, also classical computer resources can be saved by utilizing the RI approximation.

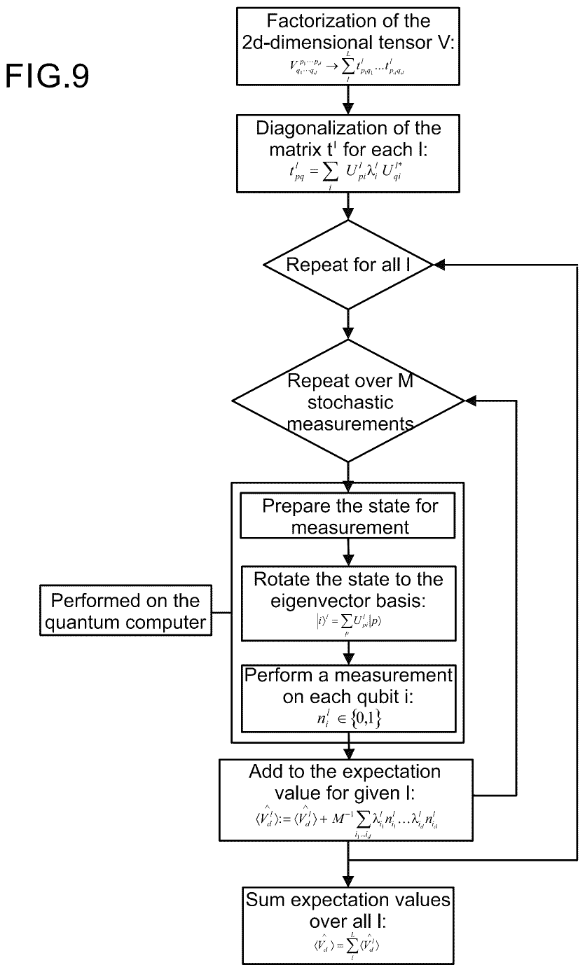

In the following detailed descriptions of preferred embodiments of the solution for the improved measurement process on quantum computers are provided, wherein a general preferred outline of a preferred method is also illustrated in FIG. 9. FIG. 9 shows a schematic and exemplary flowchart of a hybrid quantum-classical implementation of a preferred method for improving measurement processes of general electronic structure operators on a quantum computer. In this preferred embodiment the problem refers to the determination of an expectation value of the general Hermitian electronic structure operator \hat{V}_{d} with respect to a quantum state |ψ [symbol] prepared on the quantum computer (see also Eq. (1)):

$$〈 {\hat{V}}_{d} 〉 = 〈 Ψ \vert {\hat{V}}_{d} \vert Ψ 〉 \qquad ( 10 )$$

In a first step the problem comprising in this example a (relativistic) electronic structure problem as well as the physical quantity of interest that is represented by the operator \hat{V}_{d} are defined on a classical computer, for example, by respective user input. Generally, an electronic structure system is defined by the types of atoms and their coordinates as well as its electric charge and spin multiplicity. This information can be provided with the providing of the problem description and, for example, defines the chemical product to which the problem relates. Furthermore, suitable basis sets and optionally pseudopotentials representing the energetically lowest-lying energy states can be chosen as part of providing the problem. Further, the providing can also comprise determining a set of starting orthogonal molecular orbitals in the space spanned by the basis functions. Furthermore, the type of the Hamiltonian characterizing the electronic structure, usually, a non-relativistic Schrö-dinger-type Hamiltonian within the Born-Oppenheimer approximation, can be chosen, for example based on the respective chemical product that should be calculated or based on user input. The following example is focused on more general Hamiltonians, in particular Hamiltonians containing scalar-relativistic potentials, pseudo-relativistic two-component Hamiltonians describing spin-orbit coupling as well as the fully relativistic four-component Dirac-Hamiltonian. Further optional choices included in the providing of the problem can refer to choosing additional potentials in the Hamiltonian that for example describe solvation and/or environment effects, for example, via continuum solvation models like the conductor-like screening model (COSMO), external electrical fields, etc. In addition to the electronic structure system the physical quantity of interest that is represented by the operator is provided as part of the problem description. This quantity can refer, for example, to the (relativistic) Hamiltonian (see Eq. (5)) to obtain the energies, to the electrostatic multipole operator to obtain electrostatic multipole moments, etc.

In total this information provided as or with the problem can then be utilized to define as quantum mechanical representation the operator of interest, Va, by determining D (and with that also d), N and V (see Eq. (1)). Preferably, the latter quantity is determined by evaluating the matrix elements of the operator in the chosen basis set on the classical computer. Generally, the providing of the problem description can also refer to directly providing the problem description in this quantum mechanical representation, wherein in this case the transformation of the problem description only refers to determining the unitary transformations, as described in the following.

Moreover, also parameters for the calculation process itself can be provided, for example, the number of repetitions or stochastic measurements, M, which are required to reach the desired statistical accuracy and an appropriate set of auxiliary functions l with overall dimension L.

In the following details are given on how to prepare the operator of interest on a classical computer in such a way that its expectation value can be measured more efficiently on a quantum computer. In the special case of one- and two-electron operators even more efficient techniques as for general operators can be used, which will be detailed afterwards. All procedures described for complex vectors, matrices and tensors (relevant e.g. for relativistic electronic structure calculations) apply also to real vectors, matrices and tensors as a special case (relevant e.g. for non-relativistic electronic structure calculations). When performing operations on real instead of complex data, Hermitian is to be replaced with symmetric, Hermitian conjugate with transpose, and complex conjugate with the respective real value.

In the following, a preferred embodiment for determining the unitary transformations, as performed, for example, by the transformation unit, is described, for the above example problem. The 2d-dimensional complex tensor V, defined in Eq. (1), is assumed to be Hermitian with respect to the simultaneous interchange of all lower and upper indices:

$${V}_{{q}_{1} … {q}_{d}}^{{p}_{1} … {p}_{d}} = {V}_{{p}_{1} … {p}_{d}}^{{q}_{1} … {q}_{{d}^{*}}} \qquad ( 11 )$$

If this property is not fulfilled, then the procedure to construct the Hermitian component described later can be applied. Moreover, the tensor is assumed to be symmetric with respect to identical simultaneous permutations within the lower and upper indices:

$${V}_{… {q}_{i} … {q}_{k} …}^{… {p}_{i} … {p}_{k} …} = {V}_{… {q}_{k} … {q}_{i} …}^{… {p}_{k} … {p}_{i} …} \qquad ( 12 )$$

The tensor V is then preferably factorized in a first step in terms of a rank three tensor t′ on a classical computer leading to:

$${\hat{V}}_{d} = {\sum }_{l = 1}^{L} {( {\sum }_{p , q}^{N} {t}_{p q}^{l} {\hat{c}}_{p}^{†} {\hat{c}}_{q} )}_{1} … {( {\sum }_{p , q}^{N} {t}_{p q}^{l} {\hat{c}}_{p}^{†} {\hat{c}}_{q} )}_{d} \qquad ( 13 )$$

For each specific l, t^{l} is a Hermitian square matrix. Thus, in a further step it can be diagonalized on a classical computer and expressed in terms of eigenvalues

$${λ}_{i}^{l}$$

and eignvectors U^{l}:

$${t}_{p q}^{l} = {\sum }_{i = 1}^{N} {U}_{p i}^{l} {λ}_{i}^{l} {U}_{q i}^{l *} \qquad ( 14 )$$

In the case of a non- or scalar-relativistic formalism, where molecular orbitals can be described by real spin-up and spin-down orbitals, φ_{p↑} and φ_{p↓}, respectively, it is preferred that the matrices

$${t}_{p ↑ q ↑}^{l} and {t}_{p ↓ q ↓}^{l}$$

are diagonalized separately. In general, the diagonalization of the matrices

$${t}_{p ↑ q ↓}^{l} and {t}_{p ↓ q ↑}^{l}$$

can be omitted as all their elements are zero by construction. In case φ_{p↑} and φ_{p↓} have the same spatial part for all p, which can be the case within a restricted closed-shell or restricted open-shell formalism,

$${t}_{p ↑ q ↑}^{l} = {t}_{p ↓ q ↓}^{l}$$

can be determined such that a diagonalization of either one of the two matrices is sufficient.

After diagonalization inserting Eq. (14) into Eq. (13) yields:

$${\hat{V}}_{d} = {\sum }_{l = 1}^{L} {( {\sum }_{p , q}^{N} {\sum }_{i}^{N} {U}_{p i}^{l} {λ}_{i}^{l} {U}_{q i}^{l *} {\hat{c}}_{p}^{†} {\hat{c}}_{q} )}_{1} … {( {\sum }_{p , q}^{N} {\sum }_{i}^{N} {U}_{p , i}^{l} {λ}_{i}^{l} {U}_{q , i}^{l *} {\hat{c}}_{p}^{†} {\hat{c}}_{q} )}_{d} \qquad ( 15 )$$

In the next step, the eigenvectors U^{l} can be absorbed into the second-quantized operators which then leads to:

$${\hat{V}}_{d} = {\sum }_{l = 1}^{L} {\sum }_{{i}_{1} , … , {i}_{d}}^{N} {λ}_{{i}_{1}}^{l} {\hat{n}}_{{i}_{1}}^{l} … {λ}_{{i}_{d}}^{l} {\hat{n}}_{{i}_{d}}^{l} \qquad ( 16 )$$

Here îᵢ^{l} is the occupation number operator

$${( {\hat{c}}_{i}^{l} )}^{†} {\hat{c}}_{i}^{l}$$

in the basis rotated with U^{l},

$${\hat{n}}_{i}^{l} = {\sum }_{p , q}^{N} {U}_{p i}^{l} {U}_{q i}^{l *} {\hat{c}}_{p}^{†} {\hat{c}}_{q} \qquad ( 17 )$$

wherein U^{l} refers to the unitary transformations and leads to a pure occupation number representation of the operators \hat{V}_{d}.

In general, we speak of an occupation number representation if all operators can be measured concurrently on the quantum computer. Other examples of occupation number representations are fermionic operators that include only occupation number operators or products thereof. An occupation number operator in a certain basis with creation operators

$${\hat{c}}_{i}^{†}$$

and annihilation operators ĉᵢ can be given as

$${\hat{n}}_{i} = {\hat{c}}_{i}^{†} {\hat{c}}_{i} .$$

An example for such an operator is a density-density interaction

$${\sum }_{ij}^{□} {v}_{ij} {\hat{n}}_{i} {\hat{n}}_{j} .$$

In general, all operators that, after transformation onto spin systems, e.g. qubits, contain only commuting terms that can be measured concurrently on the quantum computer can be defined as being a pure occupation number representation for the context of this application. An example is any operator that contains only σ^{z} or identity Pauli operators or products thereof for all qubits, e.g.,

$${\sum }_{ij}^{□} {J}_{ij} {σ}_{i}^{z} {σ}_{j}^{z} .$$

As all occupation number operators of the pure occupation number representation above commute with each other they can be measured concurrently on the quantum computer.

As can be seen from Eqs. (16) and (17) measuring the expectation value [symbol] V_{d} [symbol] = [symbol] ψ|V_{d}|ψ [symbol] can be decomposed into the following consecutive steps to be carried out on the quantum computer: preparing the quantum state |ψ [symbol] on the quantum computer, rotating the state to the eigenvector basis via U^{l} for a specific value of l, and finally measuring all occupation numbers

$${n}_{i}^{l}$$

referring to eigenvalues of the operator

$${\hat{n}}_{i}^{l} .$$

To achieve the desired statistical accuracy for the expectation value the process has to be repeated over M stochastic measurements. Furthermore, the process has to be repeated for all l=1, . . . , L. Thus, the quantities that are necessary to prepare the quantum state and measure the expectation value need to be transferred to the quantum computer. To carry out the improved measurement process, in particular the unitary transformation U^{l} for the current value of l also needs to be transferred to the quantum computer. For this transfer, the transformation described above can be translated into a quantum algorithm description, for example, by the translation unit, and respectively generated control signals can be provided to the quantum computer as described in the following.

In the first step on the quantum computer a control signal is provided that is indicative of the preparation of the desired quantum state |ψ [symbol] in the qubit register as a result of the quantum algorithm description. This also includes hybrid quantum-classical circuits, where typically parameterized quantum states are prepared on the quantum computer which are then subject to optimization on a classical computer in order to iteratively minimize an objective function such as the (ground-state relativistic) electronic structure energy. In the case of iterative, e.g. variational, procedures the improved measurement process described here can be applied in each iteration step.

After the quantum state |ψ [symbol] has been prepared in the qubit register, control signals corresponding to unitary transformation operations are provided to rotate each of the N individual qubit states |p [symbol] (p=1, . . . , N) to the eigenvector basis via the unitary transformation U^{l} for a specific value of l:

$${\vert i 〉}^{l} = {\sum }_{p = 1}^{N} {U}_{p i}^{l} \vert p 〉 for i = 1 , … , N \qquad ( 18 )$$

In the last step on the quantum computer, control signals comprising a hardware-specific readout protocol are provided to the quantum computer to concurrently measure the eigenvalues of all N commuting occupation number operators

$${\hat{n}}_{i}^{l}$$

for a specific value of l. For each of the N qubits either 0 or 1 is returned:

$${n}_{i}^{l} ∈ \{ 0 , 1 \} for i = 1 , … , N \qquad ( 19 )$$

After the measurement of these occupation numbers the state of the qubit register collapses to the corresponding basis state

$$\vert {n}_{1}^{l} {n}_{2}^{l} … {n}_{N}^{l} 〉 .$$

After all values are collected and transferred to the classical computer the state of the qubit register can be reset to |00 . . . 0 [symbol] .

In the next steps the final expectation value [symbol] V_{d} [symbol] is evaluated on a classical computer using the measurement results from the quantum computer according to Eq. (16) and thus a solution of the problem is determined, for example, by a determination unit. In particular, for the example shown in FIG. 9 the occupation numbers nᵢ^{l} measured on the quantum computer are transferred to the classical computer, contracted with the eigenvalues λᵢ^{l} from Eq. (14) and added to the current expectation value for a given value of l according to:

$$〈 {\hat{V}}_{d}^{l} 〉 : = 〈 {\hat{V}}_{d}^{l} 〉 + \frac{1}{M} {\sum }_{{i}_{1} , … , {i}_{d}}^{N} {λ}_{{i}_{1}}^{l} {n}_{{i}_{1}}^{l} … {λ}_{{i}_{d}}^{l} {n}_{{i}_{d}}^{l} \qquad ( 20 )$$

The preparation of the quantum state on the quantum computer and the measurement are then repeated M times, with M being the number of stochastic measurements that are required to achieve the desired statistical accuracy for the expectation value [symbol] V_{d} [symbol] . This entire sub-procedure is then repeated additionally for all l=1, . . . , L.

Lastly, the expectation values determined above are then summed over all l to obtain the expectation value for [symbol] V_{d} [symbol] according to:

$$〈 {\hat{V}}_{d} 〉 = {\sum }_{l = 1}^{L} 〈 {\hat{V}}_{d}^{l} 〉 \qquad ( 21 )$$

A preferred embodiment refers to separating operators of the quantum mechanical representation representing observables to be measured into a first part comprising first operators and a second part comprising second operators allowing to further reduce the number of required measurements as well as the number of operations to rotate the qubit states to an appropriate basis. An example for this embodiment with respect to the above exemplary problem is described in the following. In this example the operators in Eq. (1) are separated into a first part comprising operators that can be measured accurately in the original basis and a second part comprising operators for which it is advantageous to be measured in the rotated basis:

$${\hat{V}}_{d} = {\hat{V}}_{d}^{orig} + {\hat{\tilde{V}}}_{d} \qquad ( 22 )$$

The first part

$${\hat{V}}_{d}^{orig} = {\sum }_{{p}_{1} , … , {p}_{d}}^{N} {V}_{{p}_{1} … {p}_{d}}^{{p}_{1} … {p}_{d}} {\hat{c}}_{{p}_{1}}^{†} {\hat{c}}_{{p}_{1}} … {\hat{c}}_{{p}_{d}}^{†} {\hat{c}}_{{p}_{d}} = {\sum }_{{p}_{1} , … , {p}_{d}}^{N} {V}_{{p}_{1} … {p}_{d}}^{{p}_{1} … {p}_{d}} {\hat{n}}_{{p}_{1}}^{orig} … {\hat{n}}_{{p}_{d}}^{orig} \qquad ( 23 )$$

$$with$$

$${\hat{n}}_{p}^{orig} = {\hat{c}}_{p}^{†} {\hat{c}}_{p}$$

can be measured accurately in the original basis, whereas the second part

$${\hat{\tilde{V}}}_{d} = {\sum }_{\begin{matrix}{p}_{1} , … , {p}_{d} , \\ {q}_{1} , … , {q}_{d}\end{matrix}}^{N} ( 1 - {δ}_{{p}_{1} {q}_{1}} … {δ}_{{p}_{d} {q}_{d}} ) {V}_{{q}_{1} … {q}_{d}}^{{p}_{1} … {p}_{d}} {\hat{c}}_{{p}_{1}}^{†} {\hat{c}}_{{q}_{1}} … {\hat{c}}_{pd}^{†} {\hat{c}}_{{q}_{d}} = {\sum }_{l = 1}^{L} {\sum }_{{i}_{1} , … , {i}_{d}}^{N} {\tilde{λ}}_{{i}_{1}}^{l} {{n}^{\hat{\sim}}}_{{i}_{1}}^{l} … {\tilde{λ}}_{{i}_{d}}^{l} {{n}^{\hat{\sim}}}_{{i}_{d}}^{l} \qquad ( 24 )$$

is measured as described above in the rotated bases defined by the unitary transformations Ũ^{l} with eigenvalues

$${\tilde{λ}}_{i}^{l} ,$$

which are obtained by a factorization of

$$( 1 - {δ}_{{p}_{1} {q}_{1}} … {δ}_{{p}_{d} {q}_{d}} ) {V}_{{q}_{1} … {q}_{d}}^{{p}_{1} … {p}_{d}}$$

rather than factorization of

$${V}_{{q}_{1 …} {q}_{d}}^{{p}_{1 …} {p}_{d}}$$

with δ_{pq} being the Kronecker delta.

In summary, as mentioned before the method, also referred to as low-rank decomposition, for an improved measurement process on a quantum computer reduces the number of non-concurrent measurements, Nₘₑₐₛ, to determine the expectation value of an operator \hat{V}_{d} from [symbol] (N^{2d}/ϵ²) to [symbol] (L/ϵ²). This is mainly achieved by concurrently measuring the expectation values of all N occupation number operators for each specific rotated basis l=1, . . . , L instead of measuring all [symbol] (N^{2d}) individual terms individually according to Eq. (1). In order to achieve an advantage in practical applications, the number of rotated bases L is preferably efficiently truncated. In the following, it will be shown in the case of one- and two-electron operators that this can be achieved in a relatively straightforward manner. Furthermore, the method is compatible with most other state-of-the-art methods to further improve the measurement process.

Overall, the method makes quantum computing usable for simulating large (relativistic) electronic structure problems (large N) and/or calculating quantities that are connected to operators that contain higher-dimensional tensors (large D) by reducing the number of required measurements on the quantum computer. Examples are the calculation of the energy levels and physicochemical properties of large electronic structure systems, such as large-sized molecules. The method works, in particular, when relativistic effects such as for example spin-orbit coupling are important. The method also works in the non-relativistic limit. Using the computed properties and/or the results of multiple energy calculations, which can be used to calculate energy differences, relevant quantities, e.g. technical application properties, for real-world application problems can be determined that are for example connected to molecules and solids. From that, recommendations can be derived for designing new materials and chemical products, improve chemical processes, tailor molecules, solids and materials to desired properties and make research activities much more efficient by reducing the number of required expensive lab and production trials.

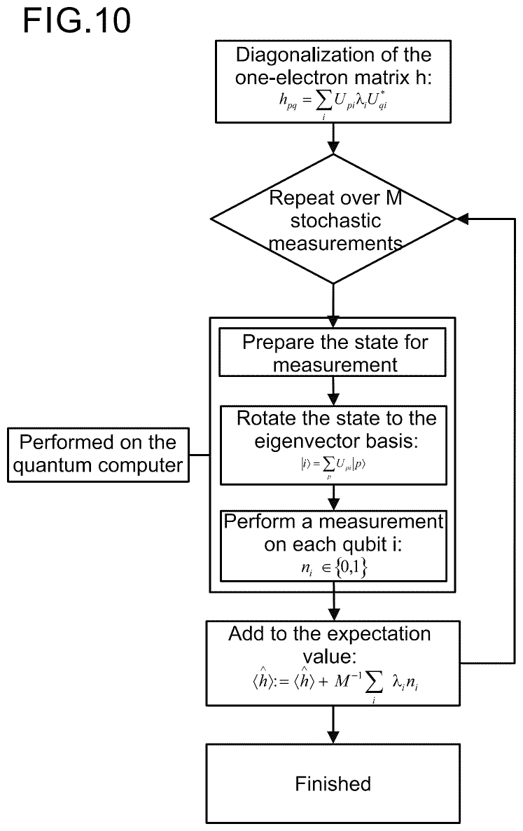

FIG. 10 schematically and exemplarily illustrates a further preferred embodiment of the invention allowing for an improved measurement of one-electron operators. The following example is focused on the modifications that can be made to efficiently measure one-electron operators on a quantum computer.

In the case of a one-electron operator the dimension is d=D=1 in the exemplary problem represented by Eq. (1). Thus, the resulting one-electron operator has the same form as the first term in the (relativistic) electronic structure Hamiltonian in Eq. (5) representing the quantum mechanical representation in this example:

$${\hat{V}}_{1} = {\sum }_{p q}^{N} {V}_{q}^{p} {\hat{c}}_{p}^{†} {\hat{c}}_{q} = {\sum }_{p q}^{N} {h}_{p q} {\hat{c}}_{p}^{†} {\hat{c}}_{q} \qquad ( 25 )$$

The number of non-concurrent measurements on the quantum computer, Nₘₑₐₛ, scales as [symbol] (N²/ϵ²) if no further improvements are made.

With the improved measurement described above the scaling can be reduced to [symbol] (1/ϵ²). In the case of one-electron operators no auxiliary basis set and no loop over I is required, i.e. L=1 in all equations above. In case the one-electron operator under consideration is the one-electron contribution to the (relativistic) Hamiltonian in Eq. (5) the integrals h_{pq} in the basis of the (molecular) orbitals are preferably determined by utilizing, e.g. a (relativistic) Hartree-Fock calculation. A result of the Hartree-Fock calculation can then also serve as a starting point for the preparation of the quantum state on the quantum computer. As no loop over I is required the factorization described above can be omitted and the matrix h can directly be diagonalized as described above. After the quantum mechanical representation is prepared the qubit states are rotated to the eigenvector basis and finally all N occupation numbers are concurrently measured on the quantum computer. As there is no loop over I this sub-process is only repeated over M stochastic measurements to obtain the final result for the expectation value [symbol] \hat{V}₁ [symbol] .

Besides contributing to the (relativistic) Hamiltonian and thereby to the energy spectrum of the electronic structure system, one-electron operators typically represent important observables that allow the prediction of physicochemical properties. Connected to one-electron operators are for example relativistic corrections, e.g. mass-velocity and Darwin corrections to the energy on the basis of a non-relativistic wavefunction, electrostatic multipole moments, magnetic moments and spin polarization, hyperfine couplings, electric fields and their gradients relevant for Mössbauer spectroscopy, diamagnetic shieldings relevant for nuclear magnetic resonance (NMR) spectroscopy. In many cases the influence of relativistic effects on those properties is crucial to enhance the understanding of the system and problem under study.

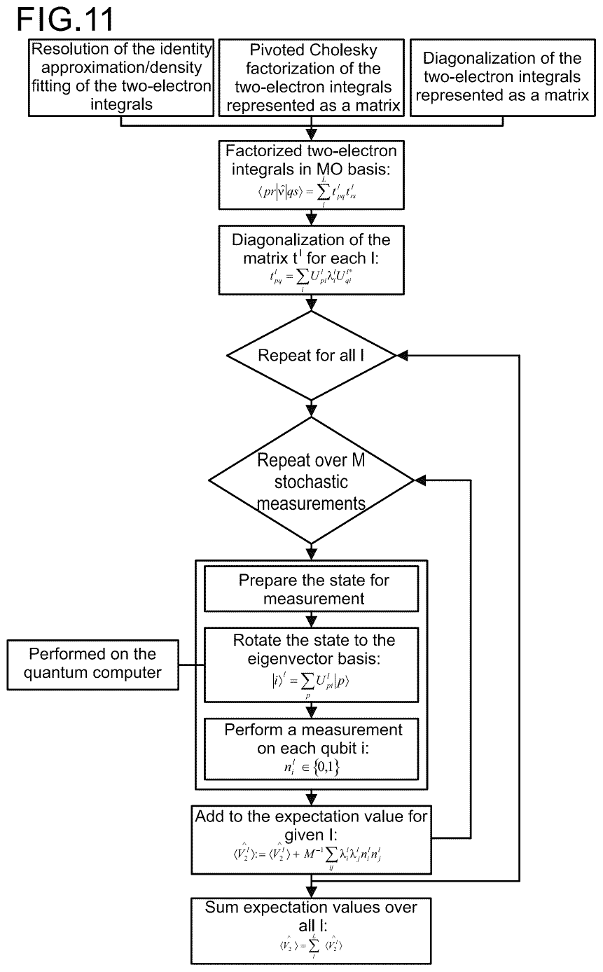

FIG. 11 schematically and exemplarily illustrates a further preferred embodiment of the invention allowing for an improved measurement of two-electron operators. In the following the description is focused on the modifications that can be made to efficiently measure two-electron operators on a quantum computer.

In the case of a two-electron operator the dimension is d=D=2 in the exemplary problem represented by Eq. (1). Thus, the resulting two-electron operator has the same form as the second term in the (relativistic) electronic structure Hamiltonian in Eq. (5) representing the quantum mechanical representation in this example:

$${\overline{V}}_{2} = {\sum }_{{p}_{1} {p}_{2} {q}_{1} {q}_{2}}^{N} {V}_{{q}_{1} {q}_{2}}^{{p}_{1} {p}_{2}} {\hat{c}}_{{p}_{1}}^{†} {\hat{c}}_{{q}_{1}} {\hat{c}}_{{p}_{2}}^{†} {\hat{c}}_{{q}_{2}} = {\sum }_{p q r s}^{N} {v}_{p q r s} {\hat{c}}_{p}^{†} {\hat{c}}_{q} {\hat{c}}_{r}^{†} {\hat{c}}_{s} \qquad ( 26 )$$

The number of non-concurrent measurements on the quantum computer, Nₘₑₐₛ, scales as [symbol] (N⁴/ϵ²), which can become a computational bottleneck if large electronic structure systems are considered and no further improvements are made.

With the improved measurement described above the scaling in general can be reduced to [symbol] (L/ϵ²) with L being the dimension of a set of rotated bases. In case the two-electron operator under consideration is the two-electron contribution to the (relativistic) Hamiltonian in Eq. (5) the two-electron integrals v_{pqrs} in the basis of the (molecular) orbitals are preferably determined by utilizing, e.g. a (relativistic) Hartree-Fock calculation. Also in this case the result of the Hartree-Fock calculation can serve as a starting point for the preparation of the quantum state on the quantum computer. If the two-electron integrals are real, the factors t^{l} are in one preferred embodiment obtained by applying a diagonalization or a pivoted Cholesky factorization, as shown in FIG. 11. In an alternative embodiment which is even more advantageous a resolution of the identity (RI) approximation of v_{pqrs}, preferably, for simplicity only considering Coulomb interaction, i.e. no Breit contribution, is used, which works for both real and complex two-electron integrals. After diagonalization of the factors on the classical computer and rewriting the two-electron (interaction) operator only in terms of density-density interaction the quantum state is prepared on the quantum computer, the qubit states are rotated to the eigenvector basis I and finally all occupation numbers are concurrently measured on the quantum computer as described above. This sub-process is then repeated over M stochastic measurements as well as for all rotated bases I and the final result for the expectation value [symbol] \hat{V}₂ [symbol] is obtained as described above. A modification of this method can include the specifics described above referring to the separation of the operators of the quantum mechanical representation representing observables to be measured into a first part comprising first operators and a second part comprising second operators to further reduce the number of required measurements as well as the number of operations to rotate the qubit states to the auxiliary basis.

The efficient measurement of the two-electron operator contributing to the (relativistic) Hamiltonian in Eq. (5) is of central importance for virtually all electronic structure calculations, as it allows the efficient determination of the energy levels of larger electronic structure problems. As an example, within a variational hybrid quantum-classical algorithm the total energy needs to be calculated in each iteration step to decide whether the desired energy state, e.g. the ground state, is reached and the iterative procedure is converged. Furthermore, calculating energy differences of different electronic states of an electronic structure system, e.g. a molecule, allows the prediction of spectroscopic properties, which are important to understand the interaction of radiation and matter, e.g. in organic electronics. Another example is chemical reactivity. Here the energy differences between educts, products and the transition state, e.g. the highest point in energy along the reaction pathway, allow for a prediction of chemical reactivity, e.g. thermodynamics and kinetics of a chemical reaction. Lastly, energy differences need to be calculated in order to predict the magneto-crystalline anisotropy of magnetic materials.

The influence of relativistic effects on the above mentioned energies and properties is preferably taken into account whenever heavy elements with high atomic numbers are involved, for example heavy main group elements, transition metals, lanthanides and actinides. In such cases, in order to obtain highly accurate results for the above mentioned energies and properties, besides a quantum mechanical description and solution of the problem it is preferred that additionally relativistic effects are accounted for. Relativistic effects generally refer to the discrepancies between solutions of models, e.g. Hamiltonian operators, that consider the theory of relativity and those that do not. Thus, in the above described cases both the theory of quantum mechanics and the theory of relativity are preferably accounted for to obtain highly accurate results. A prominent example is the explanation for the color of gold: due to relativistic effects, it is not silvery like most other metals. Relativistic effects are, for instance, relevant for permanent magnets or in catalysis where heavy elements like platinum and iridium play an important role as well as for chelating agents that interact with heavy metal ions. Relativistic effects are also preferably taken into account in many cases where only relatively light elements are involved, e.g. for describing and understanding level shifts and splittings in spectroscopy as well as for describing and understanding “spin-forbidden” transitions between different electronic states, such as phosphorescence and intersystem crossing which are both facilitated by spin-orbit coupling and are relevant for organic electronic materials.

In the following utilizing the resolution of the identity (RI) approximation as shown in the first step in FIG. 11 is described in more detail first for real two-electron integrals and then also for complex two-electron integrals. The two-electron Coulomb repulsion integrals between real orbitals φₚ, . . . in the interacting non- or scalar-relativistic Hamiltonian are defined as:

$${v}_{p q r s} = 〈 pr \vert \frac{1}{{r}_{12}} \vert qs 〉 = \int \int \frac{{ϕ}_{p} ( {\vec{r}}_{1} ) {ϕ}_{q} ( {\vec{r}}_{1} ) {ϕ}_{r} ( {\vec{r}}_{2} ) {ϕ}_{s} ( {\vec{r}}_{2} )}{\vert {\vec{r}}_{1} - {\vec{r}}_{2} \vert} d {\vec{r}}_{1} d {\vec{r}}_{2} \qquad ( A1 )$$

Resolution of the identity (RI) methods introduce an additional set of functions, the auxiliary basis set χₗ, to approximate products of two orbitals:

$${ϕ}_{p} ( \vec{r} ) {ϕ}_{q} ( \vec{r} ) \approx {\sum }_{l} {d}_{l}^{p q} {χ}_{l} ( \vec{r} ) \qquad ( A2 )$$

$${d}_{l}^{p q}$$

are the coefficients in the linear expansion for each product of two orbitals φₚ and φ_{q}.

A preferred choice for an auxiliary basis set is to use Gaussian-type basis functions. Often, sets of auxiliary basis set parameters that are tailored for specific molecular orbital basis sets can be predetermined and then stored such that, for example, the transformation unit can access these sets based on the provided problem, e.g. based on the chemical product or solid product to which the problem relates. Thus, the sets can also be applied in the context of quantum computing without extra effort. Moreover, known procedures can be utilized to generate auxiliary basis sets for molecular orbital basis sets automatically. Furthermore, also arbitrary types of basis functions can be utilized. In general, if the auxiliary basis sets are tailored to the respective problem, an error introduced by the RI approximation can be significantly smaller than the error introduced by the simulation method or the finite molecular orbital basis set and thus the error introduced by the RI approximation can be negligible.

Expanding products of orbitals as a linear combination of auxiliary functions permits the two-electron repulsion integrals to be expressed as:

$${v}_{p q r s} = {\sum }_{l m} {d}_{l}^{p q} {J}_{l m} {d}_{m}^{r s} \qquad ( A3 )$$

where J is the matrix of electron repulsion integrals between auxiliary basis functions:

$${J}_{l m} = \int \int \frac{{χ}_{l} ( {\vec{r}}_{1} ) {χ}_{m} ( {\vec{r}}_{2} )}{\vert {\vec{r}}_{1} - {\vec{r}}_{2} \vert} d {\vec{r}}_{1} d {\vec{r}}_{2} \qquad ( A4 )$$

The expression in Eq. (A3) is an approximation to the exact two-electron repulsion integrals. For simplicity, v_{pqrs} will refer to the integrals subject to the RI approximation, instead of their exact values, starting from Eq. (A3). Therefore, the equality sign “=” is used instead of the “approximately equal” sign “~” that appeared in Eq. (A2).

To make use of the RI approximation in the low-rank decomposition, preferably the matrix J is previously factorized as in the following equation:

$${\sum }_{n} {L}_{l n} {L}_{m n} = {J}_{l m} \qquad ( A5 )$$

Suitable methods that can be utilized, for example, by the transformation unit, to obtain the matrices L in Eq. (A5) include Cholesky factorization of J, or the calculation of the matrix square root

$$L = {J}^{\frac{1}{2}} .$$

Comparing with Eq. (13) reveals that the two-electron four-index integrals may be factorized in terms of three-index integrals as

$${v}_{p q r s} = {\sum }_{l} {t}_{p q}^{l} {t}_{r s}^{l} \qquad ( A6 )$$

$$with$$

$${t}_{p q}^{l} = {\sum }_{m} {d}_{m}^{p q} {L}_{m l} . \qquad ( A7 )$$

Typically, the size of an auxiliary basis set grows linearly with system size N. This implies that Nₘₑₐₛ also increases linearly with system size, because it equals the dimension of the auxiliary basis set. Therefore, the above described general method in combination with the RI approximation reduces Nₘₑₐₛ to O(N/ϵ²). An advantage over other alternatives of the method that do not utilize the RI approximation is that the factorization, e.g. by diagonalization or Cholesky factorization with pivoting, of a large matrix containing all two-electron

$${t}_{p q}^{l}$$

There are different variants of the RI method, which differ in the way that the coefficients dₗ^{pq} are obtained. Below, Coulomb fitting and other fitting schemes are described that can be utilized in respective embodiments. A variant of the RI approximation is Coulomb fitting, where the expansion coefficients are chosen to minimize the Coulomb self-repulsion of the error in the density:

$${Δ}_{p q} = {min}_{{d}^{pq}} \int \int \frac{[ {ϕ}_{p} ( {\vec{r}}_{1} ) {ϕ}_{q} ( {\vec{r}}_{1} ) - {\sum }_{l} {d}_{l}^{pq} χ ( {\vec{r}}_{1} ) ] [ {ϕ}_{p} ( {\vec{r}}_{2} ) {ϕ}_{q} ( {\vec{r}}_{2} ) - {\sum }_{l} {d}_{l}^{pq} χ ( {\vec{r}}_{2} ) ]}{\vert {\vec{r}}_{1} - {\vec{r}}_{2} \vert} d {\vec{r}}_{1} d {\vec{r}}_{2} \qquad ( A8 )$$

The coefficients that minimize Eq. (A8) satisfy the equation

$${d}_{l}^{p q} = {\sum }_{m} {( {J}^{- 1} )}_{l m} {B}_{m}^{p q} , \qquad ( A9 )$$

$$where$$

$${B}_{l}^{p q} = \int \int \frac{{ϕ}_{p} ( {\vec{r}}_{1} ) {ϕ}_{q} ( {\vec{r}}_{1} ) {χ}_{l} ( {\vec{r}}_{2} )}{\vert {\vec{r}}_{1} - {\vec{r}}_{2} \vert} d {\vec{r}}_{1} d {\vec{r}}_{2} . \qquad ( 10 )$$

In practice, the most straightforward way to factorize the electron repulsion integrals via Coulomb fitting that can be utilized, for instance, by the transformation unit, is to compute

$${t}_{p q}^{l}$$

with the following steps, which do not include an explicit computation of

$${d}_{l}^{p q} .$$

In a first step the integrals

$${B}_{l}^{p q}$$

defined in Eq. (A10) and the integrals Jₗₘ defined in Eq. (A4) are calculated. In a second step the matrix J is factorized into the matrix product LL^{T}, see Eq. (A5). Suitable methods include Cholesky factorization or the matrix square root. In a last step the factors

$${t}_{p q}^{l} = {\sum }_{m} {( {L}^{- 1} )}_{l m} {B}_{m}^{p q} \qquad ( A11 )$$

are calculated. This can be performed by executing the steps in the preceding equation, e.g. by inverting the matrix L and multiplying with each B^{pq}. However, especially if L is calculated by Cholesky factorization, a preferred numerical procedure is to solve the system of linear equations

$${\sum }_{m} {L}_{l m} {t}_{p q}^{m} = {B}_{l}^{pq} \qquad ( A12 )$$

for the coefficients

$${t}_{pq}^{m} .$$

Note that the

$${d}_{l}^{pq}$$

do not need to be calculated explicitly.

Potential modifications of the Coulomb fitting scheme are workarounds for Eq. (A9) or Eq. (A11) which are preferably utilized when the matrix J is ill-conditioned or singular.

Instead of Coulomb fitting in Eq. (A8), multiple other schemes can be utilized by the transformation unit to obtain fitted coefficients

$${d}_{l}^{pq} .$$

One possibility that can be utilized is overlap fitting. In this case, the target function to be minimized is the self-overlap of the error in the fitted density:

$${Δ}_{pq} = {min}_{{d}^{pq}} \int {[ {ϕ}_{p} ( \vec{r} ) {ϕ}_{q} ( \vec{r} ) - {\sum }_{l} {d}_{l}^{pq} {χ}_{l} ( \vec{r} ) ]}^{2} d \vec{r} \qquad ( A13 )$$

The overlap-fitted coefficients dₗ^{pq} satisfy the system of linear equations:

$${\sum }_{m} {d}_{m}^{pq} \int {χ}_{l} ( \vec{r} ) {χ}_{m} ( \vec{r} ) d \vec{r} = \int {ϕ}_{p} ( \vec{r} ) {ϕ}_{q} ( \vec{r} ) {χ}_{l} ( \vec{r} ) d {r}^{→︀} \qquad ( A14 )$$

Numerous other fitting schemes can also be utilized as alternatives to Coulomb fitting and overlap fitting, which differ by the type of metric used to obtain the coefficient

$${d}_{l}^{p q}$$

explicitly or implicitly in place of Eq. (A8) or Eq. (A13).

In particular, the RI/DF approximation is preferably utilized in the context of complex Hamiltonian elements. This is, e.g., the case as soon as fully relativistic four-component or “quasi-relativistic” two-component Hamiltonians are considered. Consider a tensor of two-electron Coulomb repulsion integrals between complex multi-component functions φₚ(\vec{r}), . . . in the interacting four-component Dirac-Coulomb Hamiltonian or the interacting two-component Hamiltonian:

$${v}_{pqrs} = 〈 pr \vert \frac{1}{{r}_{12}} \vert qs 〉 = \int \int \frac{{ϕ}_{p}^{†} ( {\vec{r}}_{1} ) {ϕ}_{r}^{†} ( {\vec{r}}_{2} ) {ϕ}_{q} ( {\vec{r}}_{1} ) {ϕ}_{s} ( {\vec{r}}_{2} )}{\vert {\vec{r}}_{1} - {\vec{r}}_{2} \vert} d {\vec{r}}_{1} d {\vec{r}}_{2} \qquad ( A15 )$$

As in the real case, the tensor can be factorized according to

$${v}_{pqrs} = {\sum }_{l} {t}_{pq}^{l} {t}_{rs}^{l} . \qquad ( A16 )$$

The complex coefficients

$${t}_{p q}^{l}$$

are products of fitting coefficients

$${d}_{l}^{p q}$$

and factors Lₗₘ, equivalent to Eq. (A7).

A preferred implementation of the method, for instance, in the transformation unit, uses real auxiliary functions χ₁(\vec{r}), . . . and determines complex fitting coefficients

$${d}_{l}^{pq} .$$

In that situation, the matrix L would be obtained analogously to the real case. Note that, as in the Coulomb fitting case for real integrals, it may be possible to determine the coefficient

$${t}_{pq}^{l}$$

directly without calculating

$${d}_{l}^{pq}$$

exlicitly. It is, however, also possible to implement the method such that the auxiliary functions χₗ(\vec{r}), . . . themselves are complex. In that case, the matrix Jₗₘ is complex symmetric, and the factors Lₗₘ can be obtained through a suitable technique, such as a Takagi factorization or Cholesky factorization for complex symmetric matrices. It is noted that in both cases the respective equations are analogous to the ones presented for real two-electron integrals above; the difference being that the complex multi-component functions φₚ and φᵣ are replaced by their Hermitian conjugates

$${ϕ}_{p}^{†} and {ϕ}_{r}^{†} ,$$

respectively, while φ_{q} and φₛ remain non-conjugated in the equations above.

Utilizing a resolution of the identity (RI) approximation, as described above, for example, for decomposition of the complex two-electron integrals v_{pqrs} in the Hamiltonian as part of the determination of the unitary transformations, is particularly advantageous as the scaling for the number of terms to be measured, e.g. non-concurrently, on a quantum computer can be significantly reduced. Additionally, existing optimized auxiliary basis sets from traditional quantum chemistry can be used that allow to omit the factorization by diagonalization to arrive at Eq. (13)

$${t}_{pq}^{l}$$

are then simply the density-fitted three-index integrals either in the basis of real molecular orbitals p, q or in the basis of complex molecular spinors p, q. In both cases the density-fitted three-index integrals can be calculated in the basis of real atomic orbitals and then preferably transformed, for example, by the transformation unit, to

$${t}_{pq}^{l}$$

either with real molecular orbital coefficients or complex molecular spinor coefficients. l are auxiliary functions. The number of auxiliary functions scales linear with the system size even without invoking further screening criteria, i.e. L∝ [symbol] (N) and thus the number of nonconcurrent measurements, Nₘₑₐₛ, can be reduced from [symbol] (N⁴/ϵ²) to [symbol] (N/ϵ²) by the above described improved measurement protocol.

The two-electron integrals may be complex, for example with some relativistic Hamiltonians. Unless the RI approximation is used to avoid the explicit factorization of v_{pqrs} it is preferred that the double factorization procedure as shown in FIG. 11 is modified. As in the real case, the first step is the formation of a super-matrix from v_{pqrs}, such that the rows of the super-matrix are labelled with the joint index pq and the columns are labelled with the joint index rs. The complex matrix is symmetric with respect to the interchange of its rows and columns: v_{pqrs}=v_{rspq}.

One possibility to factorize a symmetric complex matrix is Takagi factorization, also referred to as Autonne-Takagi factorization, as described, for example, in the book “Matrix Analysis” by R. A. Horn, C. R. Johnson, 2nd edition, Cambridge University Press, corollary 4.4.4:

$${v}_{pqrs} = {\sum }_{l} {w}_{pq}^{l} {s}_{l} {w}_{rs}^{l} \qquad ( 27 )$$

$${w}_{p q}^{l}$$

are the elements of a unitary super-matrix obtained from Takagi factorization. Its rows are labelled through the joint index pq, the columns with the index l. The entries sₗ contain the singular values of the super-matrix v_{pqrs}, which are real and non-negative. Three-index tensors

$${t}_{p q}^{l} ,$$

as per Eq (27), are obtained preferably as follows:

$${t}_{pq}^{l} = {w}_{pq}^{l} \sqrt{{s}_{l}} \qquad ( 28 )$$

Takagi factorization is a special type of a singular value decomposition. For each specific l, the matrix t^{l} is diagonalized according to Eq. (14). In the case of real two-electron integrals, Takagi factorization reduces to a diagonalization.

A second possibility to perform a factorization of a symmetric complex matrix is a counterpart of pivoted Cholesky factorization to obtain:

$${v}_{p q r s} = {\sum }_{l} {t}_{p q}^{l} {t}_{r s}^{l} \qquad ( 29 )$$

The implementation of an algorithm to perform a pivoted Cholesky factorization of a complex symmetric matrix has been described, for example, in the article “A dense complex symmetric indefinite solver for the Fujitsu AP3000”, P. Strazdins, Technical Report TR-CS99-01, Canberra 0200 ACT, Australia, 1999. http://hdl.handle.net/1885/40733.

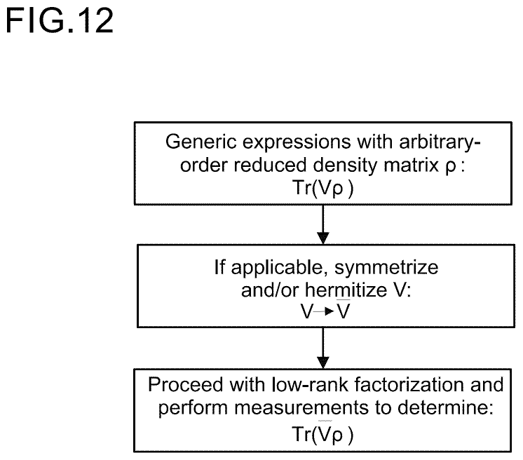

FIG. 12 illustrates an adaptation of the method described above to allow for an improved measurement of quantities connected to arbitrary-order reduced density matrices, in particular, an adaptation of the method for problems that do not lead to a symmetric, or more general Hermitian, tensor V. In the following again it will only be focused on the modifications necessary.

In general, the expectation value of the operator under consideration, see Eq. (1), can be written as a trace

$$〈 {\hat{V}}_{d} 〉 = 〈 Ψ \vert {\hat{V}}_{d} \vert Ψ 〉 = {\sum }_{{p}_{1} , … , {p}_{d} , {q}_{1} , … , {q}_{d}}^{N} {V}_{{q}_{1 …} {q}_{d} }^{{p}_{1 …} {p}_{d} d} {ρ}_{{q}_{1 …} {q}_{d}}^{{p}_{1 …} {p}_{d}} = Tr ( {V}_{}^{d} ρ ) \qquad ( 30 )$$

with the dth-order density matrix

$${}_{d}^{} {ρ}_{{q}_{1 …} {q}_{d}}^{{p}_{1 …} {p}_{d}} = ( Ψ \vert {\hat{c}}_{{p}_{1}}^{†} {\hat{c}}_{{q}_{1}} … {\hat{c}}_{{p}_{d}}^{†} {\hat{c}}_{{q}_{d}} \vert Ψ 〉 \qquad ( 31 )$$

In the general method described, for example, with respect to FIG. 9 the problem leads to the tensor V possessing the symmetries outlined in Eqs. (11) and (12). If the problem leads to a tensor V that does not possess the aforementioned symmetries, it is preferred that the method comprises introducing the symmetries. Instead of the expression in Eq. (31), the normal-ordered density

$${}_{d}^{} \ddot{ρ}$$

is utilized:

$${}_{d}^{} {\ddot{ρ}}_{{q}_{1 …} {q}_{d}}^{{p}_{1 …} {p}_{d}} = 〈 Ψ \vert {\hat{c}}_{{p}_{1}}^{†} … {\hat{c}}_{{p}_{d}}^{†} {\hat{c}}_{{q}_{d}} … {\hat{c}}_{{q}_{1}} \vert Ψ 〉 \qquad ( 32 )$$

With the appropriate normal-ordered densities, Eq. (30) can be expressed as

$$〈 {\hat{V}}_{d} 〉 = Tr ( {\ddot{V}}_{}^{d} \ddot{ρ} ) , \qquad ( 33 )$$

where \ddot{V} are the coefficients derived from V and associated with the normal-ordered density. The expressions without normal ordering (Eq. (30)) and with normal ordering (Eq. (33)) are related through the anticommutation relations of the second quantization operators:

$${c}_{p}^{†} {c}_{q}^{†} + {c}_{q}^{†} {c}_{p}^{†} = 0 \qquad ( 34 )$$

$${c}_{p} {c}_{q} + {c}_{q} {c}_{p} = 0$$

$${c}_{p}^{†} {c}_{q} + {c}_{q} {c}_{p}^{†} = {δ}_{p q}$$

First, it is preferred to establish permutational symmetry as per Eq. (12). By construction, this type of symmetry is fulfilled for a normal-ordered d-th order density

$${}_{d}^{} \ddot{ρ} .$$

Among the d indices, there are d! unique permutations [symbol] ₙ{p₁ . . . p_{d}}. Eq. (33) can thus be expressed equivalently as:

$$〈 {\hat{V}}_{d} 〉 = \frac{1}{d !} {\sum }_{n}^{d !} {\sum }_{{p}_{1} , … , {p}_{d} , {q}_{1} , … , {q}_{d}}^{N} {{\ddot{V}}_{{𝒫}_{n} \{ {q}_{1 …} {q}_{d} \}}^{{𝒫}_{n} \{ {p}_{1 …} {p}_{d} \}}}_{}^{d} {\ddot{ρ}}_{{q}_{1} {… q}_{d}}^{{p}_{1} {… p}_{d}} \qquad ( 35 )$$

Note that the order of the upper and lower indices is always defined by an identical permutation. Second, symmetry with respect to exchange of the upper and lower indices (Eq. (11)) is preferably established for real quantities by virtue of the symmetry of the density matrix:

$$〈 {\hat{V}}_{d} 〉 = \frac{1}{2} {\sum }_{{p}_{1} , … , {p}_{d} , {q}_{1} , … , {q}_{d}}^{N} ( {\ddot{V}}_{{q}_{1 …} {q}_{d}}^{{p}_{1 …} {p}_{d}} + {\ddot{V}}_{{p}_{1 …} {p}_{d}}^{{q}_{1 …} {q}_{d}} ) {}_{d}^{} {{\ddot{ρ}}_{{q}_{1 …} {q}_{d}}^{{p}_{1 …} {p}_{d}}}^{} \qquad ( 36 )$$

If [symbol] \hat{V}_{d} [symbol] is known to be real, which is the case for expectation values of operators representing observable physical quantities such as the energy, then only the Hermitian component of \ddot{V} is needed:

$${〈 {\hat{V}}_{d} 〉 = \frac{1}{2} {\sum }_{{p}_{1} , … , {p}_{d} , {q}_{1} , … , {q}_{d}}^{N} {( {\ddot{V}}_{{q}_{1 …} {q}_{d}}^{{p}_{1 …} {p}_{d}} + {\ddot{( V}}_{{p}_{1 …} {p}_{d}}^{{q}_{1 …} {q}_{d}} )}^{*} )}^{} {}_{d}^{} {\ddot{ρ}}_{{q}_{1 …} {q}_{d}}^{{p}_{1 …} {p}_{d}} \qquad ( 37 )$$

Likewise, only the imaginary part of [symbol] \hat{V}_{d} [symbol] is obtained when \ddot{V} is replaced by its anti-Hermitian component.

Generally, Eq. (33) is then preferably replaced by the equation

$$〈 {\hat{V}}_{d} 〉 = Tr ( \ddot{\overline{V}} {}_{d}^{} \ddot{ρ} ) , \qquad ( 38 )$$

where \overline{\ddot{V}} is the tensor obtained through symmetrization of \ddot{V} with respect to permutation,

$${\ddot{\overline{V}}}_{{q}_{1} {… q}_{d}}^{{p}_{1} {… p}_{d}} = \frac{1}{d !} {\sum }_{n}^{d !} {\ddot{V}}_{{𝒫}_{n} \{ {q}_{1 …} {q}_{d} \}}^{{𝒫}_{n} \{ {p}_{1 …} {p}_{d} \}} , \qquad ( 39 )$$

or by hermitizing, e.g. symmetrization in the real case, of \ddot{V} with respect to exchange of indices,

$${\ddot{\overline{V}}}_{{q}_{1} … q d}^{{p}_{1} … {p}_{d}} = \frac{1}{2} ( {\ddot{V}}_{{q}_{1} … q d}^{{p}_{1} … {p}_{d}} + {( {\ddot{V}}_{{p}_{1} … {p}_{d}}^{{q}_{1} … q d} )}^{*} ) , \qquad ( 40 )$$

or a combination of both:

$${\ddot{\overline{V}}}_{{q}_{1} … q d}^{{p}_{1} … {p}_{d}} = \frac{1}{2 d !} {\sum }_{n}^{d !} ( {\ddot{V}}_{{𝒫}_{n} \{ {q}_{1} … q d \}}^{{𝒫}_{n} \{ {p}_{1} … {p}_{d} \}} + {( {\ddot{V}}_{{𝒫}_{n} \{ {p}_{1} … {p}_{d} \}}^{{𝒫}_{n} \{ {q}_{1} … q d \}} )}^{*} ) . \qquad ( 41 )$$

Using the anticommutation relations for the second-quantized creation and annihilation operators or by other algebraic means, the symmetrized tensor \overline{\ddot{V}} is transformed to the nonnormal-ordered form \hat{V} that is suitable for factorization.

Finally, the expectation value of the operator is expressed in terms of V̅ as in Eq. (30):

$$〈 {\hat{V}}_{d} 〉 = T r ( {V}_{}^{d} ρ ) = T r ( {\overline{V}}_{}^{d} ρ ) . \qquad ( 42 )$$

After this the method proceeds with processing of V̅, as already described above, for instance, with respect to FIGS. 9 to 11, for improved measurement of the expectation value [symbol] \hat{V}_{d} [symbol] . A modification of the method could include the variations described above.

With any of the methods described above the number of non-concurrent measurements on the quantum computer, Nₘₑₐₛ, to determine [symbol] \hat{V}_{d} [symbol] can be reduced from [symbol] (N^{2d}/ϵ²) to [symbol] (L/ϵ²) which potentially overcomes a severe computational bottleneck, in particular for quantities for which d>2, when combined with an appropriately truncated set of auxiliary functions of dimension L. The measurement of higher-order reduced density matrices can be important for perturbative methods that can enhance the overall accuracy of the result, e.g. the energy, by an additional post-processing step on a classical computer if the exact solution of the full electronic structure problem cannot be prepared on the quantum computer. Examples for such methods are second-order n-electron valence state perturbation theory (NEVPT2) and second-order complete active space perturbation theory (CASPT2) that lead to third- and fourth-order reduced density matrices, i.e. d=3 and 4 resulting in [symbol] (L/ϵ²) instead of [symbol] (N⁸/ϵ²) non-concurrent measurements on the quantum computer when using the above described improved measurement method. Furthermore, a quantum subspace expansion method, as described, for instance, in the article “Hybrid quantum-classical hierarchy for mitigation of decoherence and determination of excited states” McClean et al. Phys. Rev. A 95, 042308 (2017), that can be generally used to determine electronically excited states and/or mitigate decoherence, also requires the measurement of higher-order reduced density matrices, i.e. d>2. Thus, it can also be made more efficient by the above described measurement method.

The above described method and its embodiments can also be applied to other problems than electronic structure problems described exemplarily above, such as mixed fermion-boson systems, e.g. electron-photon (radiation-matter interaction), electron-phonon, etc. problems. Also, the method can be applied within the computation of nuclear wave functions or mixed nuclear/electronic wave functions.

Generally, the above described invention improves known methods and algorithms by improving the measurement, in particular, such that it can also be applied to operators that are present in problems utilizing relativistic electronic structure problem formulations. This is in particular advantageous for the above described applications. Further, in contrast to known methods the above described invention preferably utilizes the resolution of the identity approximation for improving the measurement of the two-electron terms in a Hamilton operator formulation of a problem, which allows an optimal reduction of the measurement effort while minimizing the approximation errors.

Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the providing of the problem description, the transformation of the problem description, the translation of the quantum mechanical representation, the generating of the control signals, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refers to an apparatus for generating control signals for measuring states of quantum elements of a quantum computer. A providing unit provides a problem description. A transformation unit transforms the problem description into a quantum mechanical representation The transformation comprises determining unitary transformations that rotate operators representing observables to be measured into bases that lead to a pure occupation number representation. A translation unit translates the quantum mechanical representation into a sequence of quantum operations comprising unitary transformation operations to be applied to quantum elements. The translating comprises determining the unitary transformation operations based on the determined unitary transformations. A generation unit generates control signals for controlling the application of the determined sequence of quantum operations to the quantum computer such that the quantum mechanical representation of the problem is prepared and the observables indicative of the solution of the problem are measured.

1. An apparatus for generating control signals for measuring states of quantum elements of a quantum computer, wherein the measured states are indicative of observables indicative of a solution of a problem being translatable into a quantum mechanical description, wherein the apparatus comprises:
a problem providing unit for providing a problem description indicative of the problem to be solved, wherein the problem description is translatable into a quantum mechanical description,
a transformation unit for transforming the problem description into a quantum mechanical representation comprising one or more operators representing one or more observables to be measured indicative of the solution of the problem, wherein the transformation further comprises determining unitary transformations that rotate the one or more operators representing one or more observables to be measured into one or more bases that lead to a pure occupation number representation of the one or more operators after application of the unitary transformations,
a translation unit for translating the quantum mechanical representation into a quantum algorithm description comprising a sequence of quantum operations to be applied to quantum elements of the quantum computer, wherein the sequence of quantum operations comprises a) a preparation part comprising quantum operations for preparing the quantum mechanical representation on the quantum computer such that the observables being indicative of the solution of the problem are measurable, and b) a measuring part comprising quantum operations for measuring the observables by measuring the states of the quantum elements after the preparation of the quantum mechanical representation, wherein the measurement operations comprise unitary transformation operations to be applied to quantum elements, wherein the translating comprises determining the unitary transformation operations based on the determined unitary transformations such that the unitary transformation operations initiate a rotation of the states of the quantum elements into respective basis states corresponding to the one or more bases leading to the pure occupation number representation of the one or more operators representing the one or more observables to be measured, and
a control signal generation unit for generating control signals for controlling the application of the determined sequence of quantum operations to the quantum computer such that the quantum mechanical representation of the problem is prepared and the observables indicative of the solution of the problem are measured according to the quantum algorithm description.

2. The apparatus according to claim 1, wherein the transformation unit is adapted to determine the unitary transformations by applying i) a factorization to a tensor representation of one or more operators of the one or more observables to be measured and ii) a diagonalization to the resulting matrix terms of the factorized tensor representation of the one or more operators.

3. The apparatus according to claim 2, wherein the transformation unit is adapted, for the diagonalization of the matrix terms in the factorized tensor representation of the one or more operators, to determine, for each matrix term of the factorized tensor representation, eigenvalues and corresponding eigenvectors and to determine the to be measured observables as an expression in terms of corresponding eigenvectors and eigenvalues of the factorized tensor representations of the operators.

4. The apparatus according to claim 1, wherein the problem description is translatable into a relativistic Hamiltonian description of the problem.

5. The apparatus according to claim 4, wherein the transformation unit is adapted to apply a Takagi factorization or a pivoted Cholesky factorization for determining the unitary transformations.

6. The apparatus according to claim 1, wherein the transformation unit is adapted to separate operators of the quantum mechanical representation representing observables to be measured into a first part comprising first operators and a second part comprising second operators, wherein the transformation unit is adapted to transform the second operators into a quantum mechanical representation comprising only a pure occupation number representation of the operators representing the one or more observables to be measured.

7. The apparatus according to claim 1, wherein the quantum mechanical representation comprises two-electron operators representing observables to be measured wherein the transformation unit is adapted to transform the two-electron operators into terms of a density-density interaction.

8. The apparatus according to claim 7, wherein the transformation unit is adapted to transform the two-electron operators into terms of a density-density interaction by utilizing a resolution of the identity approximation.

9. The apparatus according to claim 1, wherein the quantum mechanical representation comprises operators referring to an arbitrary-order reduced density matrix and an arbitrary-order tensor, wherein the transformation unit is adapted to symmetrize and/or hermitize the arbitrary-order tensor before determining the unitary transformations.

10. A system for performing a quantum mechanical calculation on a quantum computer, wherein the system comprises:
a quantum computer adapted to perform quantum mechanical calculations based on provided control signals, and
an apparatus according to claim 1 adapted to provide control signals to the quantum computer for controlling the performance of a quantum mechanical calculation.

11. A computer implemented method for generating control signals for measuring states of quantum elements of a quantum computer, wherein the measured states are indicative of observables indicative of a solution of a problem being translatable into a quantum mechanical description, wherein the method comprises:
providing a problem description indicative of the problem to be solved, wherein the problem description is translatable into a quantum mechanical description,
transforming the problem description into a quantum mechanical representation comprising one or more operators representing one or more observables to be measured indicative of the solution of the problem, wherein the transformation further comprises determining unitary transformations that rotate the one or more operators representing one or more observables to be measured into one or more bases that lead to a pure occupation number representation of the one or more operators after application of the unitary transformations,
translating the quantum mechanical description into a quantum algorithm description comprising a sequence of quantum operations to be applied to quantum elements of the quantum computer, wherein the sequence of quantum operations comprises a) a preparation part comprising quantum operations for preparing the quantum mechanical representation on the quantum computer such that the observables being indicative of the solution of the problem are measurable, and b) a measuring part comprising quantum operations for measuring the observables by measuring the states of the quantum elements after the preparation of the quantum mechanical representation, wherein the measurement operations comprise unitary transformation operations to be applied to quantum elements, wherein the translating comprises determining the unitary transformation operations based on the determined unitary transformations such that the unitary transformation operations initiate a rotation of the states of the quantum elements into respective basis states corresponding to the one or more bases leading to the pure occupation number representation of the one or more operators representing the one or more observables to be measured, and
providing control signals for controlling the application of the determined sequence of quantum operations to the quantum computer such that the quantum mechanical representation of the problem is prepared and the observables indicative of the solution of the problem are measured according to the quantum algorithm description.

12. A computer program product for generating control signals for measuring a state of quantum elements of a quantum computer, wherein the computer program product comprises program code means for causing an apparatus 1 to execute the method according to claim 11.

13. A solution apparatus for determining a solution of a problem being translatable into a quantum mechanical description, wherein the apparatus comprises:
an apparatus according to claim 1 for generating control signals for controlling a quantum computer,
a quantum computer interface unit for interfacing with a quantum computer for providing the control signals to the quantum computer and receiving a result for the to be measured observables, and
a determination unit configured for determining a solution of the problem based on the received measurement results.

14. A property determination apparatus for determining a technical application property of a chemical product or solid product based on a solution of a problem related to the chemical product or solid product, wherein the problem is translatable into a quantum mechanical description, wherein the apparatus comprises:
an apparatus according to claim 1 for generating control signals for controlling a quantum computer,
a quantum computer interface unit for interfacing with a quantum computer for providing the control signals to the quantum computer and receiving a result for the to be measured observables, and
one or more processors configured for determining the technical application property of the chemical product or solid product based on the received measurement results.

15. An apparatus for determining a target chemical product or solid product comprising a target technical application property, wherein the apparatus comprises:
an input interface configured to provide a target technical application property and a potential chemical product or solid product,
an apparatus according to claim 1 for generating control signals for controlling a quantum computer based on the potential chemical product or solid product and based on the target technical application property,
a quantum computer interface unit for interfacing with a quantum computer for providing the control signals to the quantum computer and receiving a result for the to be measured observables,
one or more processors configured for
a) determining the technical application property of the potential chemical product or solid product based on the received measurement results, and
b) comparing the determined technical application property of the potential chemical product or solid product with the target technical application property and, based on the comparison, either i) determining the potential chemical product or solid product as the target chemical product or solid product, or ii) providing a new potential chemical product or solid product and repeating the determination of the technical application property utilizing the new potential chemical product or solid product, and
an output interface configured to provide control signals for producing the determined target chemical product or solid product.
